(19) United States
(12) Patent Application Publication
(10) Pub. No.: US 2024/0024231 A1
(43) Pub. Date: Jan. 25, 2024

(54) Hydrogel material

(71) Applicant: Standard Millox Pharmaceuticals Oy, Turku (FI)

(72) Inventors: Mika Jokinen, Turku (FI); Alar Kaljukivi, Turku (FI); Ari Rosling, Turku (FI); Tatu Assmuth, Turku (FI); Vesna Blazevic, Tampere (FI); Vesa Hytönen, Ruutana (FI); Olli Laitinen, Akaa (FI); Suvi Heinimäki, Tampere (FI)

(21) Appl. No.: 18/029,927

(22) PCT Filed: Sep. 29, 2021
(86) PCT No.: PCT/FI2021/050641

§ 371 Date: Apr. 3, 2023

(30) Foreign Application Priority Data
Oct. 1, 2020 (FI) 20205956

(51) Int. Cl. A61K 9/06; A61K 47/34; A61K 47/02

(52) U.S. Cl. CPC A61K 9/06; A61K 47/34; A61K 47/02

(57) ABSTRACT

The present invention relates to a hydrogel material hydrogel material, comprising a first hydrogel comprising 0.15-21 wt-% of a functionalised triblock molecule having a formula (1)

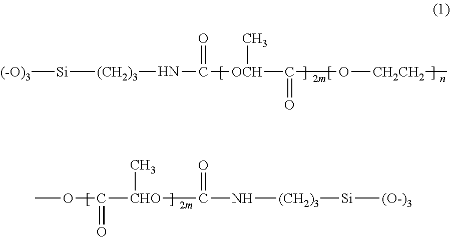

wherein n is 4-680 and m is 1-10, based on a total weight of the first hydrogel. The first hydrogel further comprises 0.85-3.3 wt-% of silica, based on the total weight of the first hydrogel, and 75-99 wt-% of an aqueous liquid, based on the total weight of the first hydrogel. The —Si—OH groups of the silica form a —Si—O—Si— bond with the —Si—(O)₃— group of the functionalised triblock molecule of formula (1).

### FIELD OF THE INVENTION

The present disclosure relates to hydrogel materials. The present hydrogel materials are especially useful for controlled delivery of biologically active agents.

### BACKGROUND

A hydrogel comprises typically a network of polymer chains or aggregated colloids as a continuous solid phase, and water as the dispersed liquid phase. A three-dimensional solid results from the hydrophilic polymer chains being held together by cross-links, or it is formed from colloids, e.g., colloidal particles due to aggregation. Hydrogels are natural or synthetic polymeric networks, where the liquid phase is water. They can be used as scaffolds in tissue engineering, as drug or cell carriers as well as sustained-release drug delivery systems.

In the present description, the following terms are used in the following meanings.

A sol is a flowing and homogeneous mixture of at least one liquid phase and one solid phase, i.e., a colloidal dispersion, where the liquid phase(s), e.g., water, ethanol and residuals of precursors, is the continuous phase and the solid phase(s), e.g. here triblock molecule, functionalised triblock and silica, in the form of colloidal particles or macromolecules, which are homogeneously dispersed in the said liquid phase.

A gel is a non-flowing homogeneous mixture of at least one solid phase and one liquid phase, i.e., a colloidal dispersion, where solid phase(s), e.g. here triblock molecule, functionalised triblock and silica form the continuous solid phase, and liquid e.g., water, ethanol and residuals of solid phase precursors, is homogeneously dispersed in the continuous solid phase. The solid phase is typically an aggregated or crosslinked molecular structure formed due to chemical reactions (e.g., by polymerisation such as polycondensation), or which is formed due to aggregation of colloids, such as nanoscale particles. In a gel, the aggregated and/or crosslinked solid phase forms a continuous network throughout the defined space, and the liquid phase is homogeneously distributed in the continuous solid network.

Sol-gel transfer is a term that refers to a process where a sol turns to a gel. The sol-gel transfer occurs typically when colloidal particles and/or macromolecules aggregate, aggregates grow in size and finally the sol turns to a gel, where the aggregates form a continuous solid phase throughout the volume of the system without phase separation of the liquid phase, i.e., the liquid phase remains homogeneously dispersed in the continuous solid phase.

A hydrogel is a gel, where the liquid phase is water or water-based containing more than 50 weight-% of water. A continuous hydrogel is a hydrogel in one three-dimensional structure. A dispersed hydrogel is a set of hydrogel particles formed by breaking a continuous three-dimensional hydrogel into smaller hydrogel particles, and in a typical case, said hydrogel particles are mixed with another, continuous hydrogel.

A flowing mixture, in the context of this invention, refers to a material, e.g., to a sol that has viscoelastic properties due to a solid and a liquid phase, but in which the flowing liquid phase dominates over the solid phase. A non-flowing mixture, in the context of this invention, refers to a viscoelastic material, e.g., to a hydrogel which has no flow properties at rest because the solid phase dominates over the liquid phase. For a flowing mixture, the viscous properties (indicated by G″, viscous/loss modulus, determined by rheological methods) dominate over the elastic properties (indicated by G′, elastic/storage modulus). Analogically, for a non-flowing mixture, the elastic properties (G′) dominate over viscous properties (G″). G′ and G″ can be measured with oscillation measurements with a rheometer with, e.g., a cone-plate or plate-plate geometry within the linear viscoelastic region under small angle oscillatory shear.

A hybrid material, in the context of this invention, is a material comprising two or more components, e.g., a triblock molecule and silica, and, in which there is a chemical bond between the components. Furthermore, in the present description, when the term hybrid is used, it refers to the solid phase comprising at least two components, which together form the solid phase in the hydrogel.

Vol-% stands for volume percentage and wt-% for weight percentage.

Nanoparticles (having a size of typically 1-100 nm) and colloids (having a size of typically 1-1000 nm) have an overlapping dimension in the range of 1-100 nm. However, they have different chemical and physical properties. Nanoparticles usually have different material properties compared to the same material (i.e. material having exactly the same chemical composition) in larger dimensions, for example their surface properties can be different, which in turn may lead to different properties when it comes to hydrophobicity or electrical conductivity. The colloids in the size of 100-1000 nm start to lose the special chemical properties of the nanoparticles as gravitation is still weaker than Brownian motion. Therefore, Brownian motion has a significant impact when various dispersions, such as gels, suspensions, emulsion and foams are manufactured, and the aim is to obtain stable dispersions.

Lyophobic colloid is a term used for colloidal dispersions, when the colloids are not thermodynamically stable (although they can be stable for 15 or even 100 years). Lyophilic colloids are thermodynamically stable but are more rarely used.

A nanoparticle or a lyophobic colloid usually refers to a state, where a certain kind of cluster of molecules has reached a size and chemical structure, that it is no longer really soluble in the surrounding liquid. However, because the nanoparticle or colloid is very small in size, it remains homogenously distributed in the surrounding liquid, even though its density can be higher than that of the liquid. This is due to the Brownian motion being stronger than gravitation. Thus, nanoparticles and colloids may, when moving in a liquid, impact each other and form a larger structure. They can for example aggregate, sometimes it is even said they are polymerised. Colloidal particles are thus aggregates formed via chemical bonds and weaker interactions (such as van der Waals interactions), while particles in the present description are formed when a three-dimensional gel has been mechanically broken down to form particles, thus comprising therein colloidal particles that have aggregated.

Document “Radiopaque Organic-Inorganic Hybrids Based on Poly(D,L-lactide)”, Mazzocchetti et al., Biomacromolecules 2007, 8, 672-678 discloses hybrid organic-inorganic nanocomposites prepared starting from α,ω-triethoxysilane-terminated poly(D,L lactic acid), to be used as potential radiopaque biocompatible coatings for medical devices. The components of the composite and the composite itself are not all water soluble. Furthermore, in this publication, tetraethyl orthosilicate (TEOS) is directly added to the process.

### OBJECTS AND SUMMARY

It is an aim to provide a hydrogel that is suitable for controlled delivery of biologically active agents, and especially useful for protective encapsulation of various biologically active agents. One particular target is encapsulation to ensure thermal stability of the biologically active agent. Another particular target is to provide a hydrogel material that can encapsulate biologically active agents while being injectable

The present description thus relates to a hydrogel material, comprising a first hydrogel comprising
0.15-21 wt-% of a functionalised triblock molecule having a formula (1)

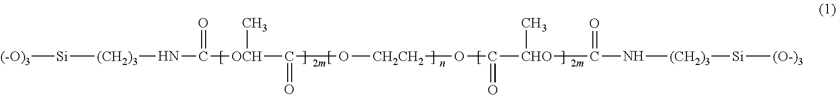

wherein n is 4-680 and m is 1-10, based on a total weight of the first hydrogel,
0.85-4.0 wt-% of silica, based on the total weight of the first hydrogel, and
75-99 wt-% of an aqueous liquid, based on the total weight of the first hydrogel,
   wherein the —Si—OH groups of the silica form a —Si—O—Si— bond with the —Si—(O)₃— group of the functionalised triblock molecule of formula (1).

The present description also relates to use of the hydrogel material for controlled delivery of a biologically active agent and for protective encapsulation of a biologically active agent.

### BRIEF DESCRIPTION OF THE DRAWINGS

FIG. 1 schematically illustrates the structure of a functionalised triblock molecule.

FIG. 2 schematically illustrates silica nanoparticle aggregates in a silica sol.

FIG. 3 schematically shows a structure of a reaction product between silica and the functionalised triblock molecule, according to an embodiment.

FIG. 4 illustrates a structure of the hydrogel material, according to an embodiment.

FIG. 5A illustrates a structure of the hydrogel material, according to another embodiment.

FIG. 5B illustrates a structure of the hydrogel material, according to yet another embodiment.

FIG. 6A shows cumulative release of antigen (norovirus P-particle) for continuous hybrid/composite hydrogel R217-02 and for two versions of two-phase hybrid/composite hydrogel #1 (R217-0.2/R40-0.2 A and R217-0.2/R40-0.2 B) at day 4 and at day 330 after storage at room temperature (25° C.)

FIG. 6B shows cumulative release of silica.

FIG. 7 shows cumulative release of antigen (norovirus P-particle) and dissolution of silica for two-phase hybrid/composite hydrogel #2 C

FIG. 8 shows cumulative release of antigen (norovirus P-particle) and dissolution of silica for two-phase hybrid/composite hydrogel #2D

FIG. 9 shows cumulative release of eGFP and dissolution of silica for continuous hybrid/composite R217-0.2 with 10 μg of eGFP in 100 μl of hydrogel

FIG. 10 shows cumulative release of eGFP and dissolution of silica for continuous hybrid/composite R217-0.2 with 20 μg of eGFP in 100 μl of hydrogel

FIG. 11 shows cumulative diffusion of eGFP from continuous hybrid/composite R217-0.2 with eGFP concentration of 10 μg/100 μl of hydrogel.

FIG. 12 shows cumulative diffusion of eGFP from continuous hybrid/composite R217-0.2 with eGFP concentration of 20 μg/100 μl of hydrogel.

FIG. 13 shows damping factor (G″/G′) for continuous hybrid/composite hydrogel R217-0.2 and for two-phase hybrid/composite hydrogel #1 (R217-0.2/R40-0.2) at day 4 and at day 28 after storage in syringes enclosed in an aluminium foil bags at room temperature (25° C.)

FIG. 14 shows damping factor (G″/G′) for continuous hybrid/composite hydrogel R217-0.2 and for two-phase hybrid/composite hydrogel #1 (R217-0.2/R40-0.2) at day 360 after storage at room temperature (25° C.)

FIG. 15 shows dynamic viscosity for continuous hybrid/composite hydrogel R217-0.2 and for two-phase hybrid/composite hydrogel #1 (R217-0.2/R40-0.2) at day 4 after storage at room temperature (25° C.).

FIG. 16 shows dynamic viscosity for continuous hybrid/composite hydrogel R217-0.2 and for two-phase hybrid/composite #1 (R217-0.2/R40-0.2) at day 360 after storage in sealed aluminium foil bags at room temperature.

FIG. 17A shows thermal stability of norovirus P-particles measured with dynamic light scattering (DLS), of a proportion of 16 nm particles at different temperatures.

FIG. 17B shows the difference in the distribution by volume for P-particles measured at 50° C. and 55° C.

FIG. 18A shows total volume of 16 nm population of norovirus P-particles released from continuous hybrid/composite hydrogel R217-0.2 kept at solution at indicated temperatures as determined with DLS.

FIG. 18B shows total volume of 16 nm population of norovirus P-particles released from control particles kept at solution at indicated temperatures as determined with DLS.

FIG. 19 shows TEM images of norovirus P-particles dissolved from hydrogels.

FIG. 20A shows kinetics of serum IgG antibodies in mice following immunisation with two 10 μg doses of norovirus P-particles (pp) alone or formulated with continuous hybrid/composite R217-0.2 hydrogel.

FIG. 20B shows kinetics of serum IgG antibodies in mice following immunisation with a single 20 μg dose of norovirus P-particles alone or formulated with two-phase hybrid/composite #1 R217-0.2/R40-0.2 hydrogel.

FIG. 21A shows endpoint titration of serum IgG in mice following immunisation with two 10 μg doses of norovirus P-particles (pp) alone or formulated with continuous hybrid/composite R217-0.2 hydrogel.

FIG. 21B shows endpoint titration of serum IgG in mice following immunisation with a single 20 μg dose of norovirus P-particles alone or formulated with two-phase hybrid/composite #1 R217-0.2/R40-0.2 hydrogel.

FIG. 22A shows endpoint titration of serum IgG1 in mice following immunisation with two 10 μg doses of norovirus P-particles (pp) alone or formulated with continuous hybrid/composite R217-0.2 hydrogel.

FIG. 22B shows endpoint titration of serum IgG1 in mice following immunisation with a single 20 μg dose of norovirus P-particles alone or formulated with two-phase hybrid/composite #1 R217-0.2/R40-0.2 hydrogel.

FIG. 23A shows endpoint titration of serum IgG2a in mice following immunisation with two 10 μg doses of norovirus P-particles (pp) alone or formulated with continuous hybrid/composite R217-0.2 hydrogel.

FIG. 23B shows endpoint titration of serum IgG2a in mice following immunisation with a single 20 μg dose of norovirus P-particles alone or formulated with two-phase hybrid/composite #1 R217-0.2/R40-0.2 hydrogel.

FIG. 24A shows avidity of serum IgG antibodies in mice immunised with two 10 μg doses of norovirus P-particles (pp) alone or formulated with continuous hybrid/composite R217-0.2 hydrogel.

FIG. 24B shows avidity of serum IgG antibodies in mice immunised with a single 20 μg dose of norovirus P-particles alone or formulated with two-phase hybrid/composite #1 R217-0.2/R40-0.2 hydrogel.

FIG. 25A shows cross-reactive serum IgG responses against heterologous NoV VLPs in mice immunised with two 10 μg doses of norovirus P-particles (pp) alone or formulated with continuous hybrid/composite R217-0.2 hydrogel.

FIG. 25B shows cross-reactive serum IgG responses against heterologous NoV VLPs in mice immunised with a single 20 μg dose of norovirus P-particles alone or formulated with two-phase hybrid/composite #1 R217-0.2/R40-0.2 hydrogel.

FIG. 26A shows homologous blockage of GII.4 VLP binding to HBGA receptors by serum antibodies of mice immunised with two 10 μg doses of norovirus P-particles (pp) alone or formulated with continuous hybrid/composite R217-0.2 hydrogel.

FIG. 26B shows homologous blockage of GII.4 VLP binding to HBGA receptors by serum antibodies of mice immunised with a single 20 μg dose of norovirus P-particles alone or formulated with with two-phase hybrid/composite #1 R217-0.2/R40-0.2 hydrogel.

FIG. 27A shows endpoint titrations of faecal IgG antibodies in mice immunised with two 10 μg doses of norovirus P-particles (pp) alone or formulated with continuous hybrid/composite R217-0.2 hydrogel.

FIG. 27B shows endpoint titrations of faecal IgG antibodies in mice immunised with a single 20 μg dose of norovirus P-particles alone or formulated with with two-phase hybrid/composite #1 R217-0.2/R40-0.2 hydrogel.

### DETAILED DESCRIPTION

The present description relates to a hydrogel material, comprising a first hydrogel comprising
0.15-21 wt-% of a functionalised triblock molecule having a formula (1)

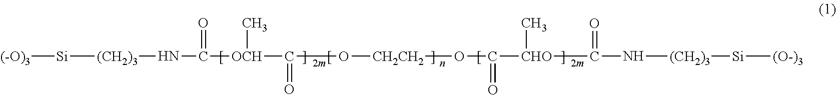

wherein n is 4-680 and m is 1-10, based on a total weight of the first hydrogel,
0.85-4.0 wt-% of silica, based on the total weight of the first hydrogel, and
75-99 wt-% of an aqueous liquid, based on the total weight of the first hydrogel,
   wherein the —Si—OH groups of the silica form a —Si—O—Si— bond with the —Si—(O)₃— group of the functionalised triblock molecule of formula (1).

The present hydrogel material has several useful properties in various fields. It has been found to be highly suitable for controlled delivery of biologically active agents. Some especially suitable biologically active agents usable with the present hydrogel material are vaccine antigens and active pharmaceutical ingredients. The hydrogel material can also be made into an injectable form, which facilitates its administration. The rheological properties of the hydrogel material can thus be tailored in a wide range, allowing tailoring of its injectability. By injectable, it is meant for example products that can be injected through a thin needle, such as 25-27 G needle, or 23-30 G needle, most typically 25-30 G needles. The present hydrogels, irrespective of the amount of aqueous liquid therein, are non-flowing when at rest, for example when stored. However, when the amount of aqueous liquid is high enough, for example 96-99 wt-%, the hydrogel becomes flowing and injectable, when a pressure is applied on it, for example by a piston of a syringe to inject the hydrogel through a needle. Such hydrogels are thus shear thinning.

The present material and all its components are water-soluble. The liquid in the present hydrogel is aqueous, which is important especially for the administering of immunomodulatory agents and therapeutically active agents. Indeed, in such uses, it is necessary that the biological activity of the agent is retained during manufacturing of the end product as well as in the final product. Furthermore, during delivery of the agent in the body, the release of the agent is mainly controlled by the biological degradation of the hydrogel material. This biological degradation mainly occurs through dissolution into the water phase of the tissue fluid. All the components of the present hydrogel dissolve in the water phase of the tissue fluid. The polymers also degrade via enzymatic reactions, the enzymes being also active in the water phase.A particularly interesting advantage is that encapsulation of biologically active agent within the present hydrogel material ensures their thermostability, as a function of storage time and temperature, i.e. their biological activity can be preserved over extended periods of time. The hydrogel material also allows a constant release rate of the biologically active agent, once administered.

The functionalised triblock molecule may also be called a hybrid, an oligomer or a polymer. It is end-capped with the silica. The —Si—O—Si— bond between the —Si—OH groups of the silica and the —Si—(O)₃— group of the functionalised triblock molecule of formula (1) are chemical bonds. In this description, the material is also called in general a hybrid/composite, as some of the materials are hybrids while others are in the form of a composite.

The hydrogel material is one continuous hydrogel, or it may consist of dispersed hydrogel particles homogeneously distributed in the continuous hydrogel.

In the above formula (1), n is 4-680 and m is 1-10. The value for n may be (independently from the value of m) for example from 4, 5, 6, 7, 8, 9, 10, 15, 20, 25, 30, 35, 40, 45, 50, 55, 60, 65, 70, 75, 80, 85, 90, 100, 120, 135, 150, 200, 225, 270, 300, 350, 400, 450, 500, 550 or 600 up to 5, 6, 7, 8, 9, 10, 15, 20, 25, 30, 35, 40, 45, 50, 55, 60, 65, 70, 75, 85, 90, 100, 120, 135, 150, 200, 225, 270, 300, 350, 400, 450, 500, 550, 600 or 680. The value for m may be (independently from the value of n) for example from 1, 2, 3, 4, 5, 6, 7 or 8 up to 2, 3, 4, 5, 6, 7, 8, 9 or 10.

The present hydrogel material may thus have various forms, which are summarised in Table 1 below. The type-names indicated in Table 1 are used in this description.

The different types of hydrogel materials are discussed in more detail below.

TABLE 1

| Type of hydrogel | | |
|---|---|---|
| material | Hydrogel structure | Components |
| Continuous | Continuous hydrogel | Functionalised triblock |
| hybrid/composite | composed of | molecule-silica |
| | hybrid/composite and | hybrid/composite and |
| | aqueous liquid | aqueous liquid |
| Two-phase | Dispersed hydrogel | Both dispersed and |
| hybrid/composite # 1 | particles homogenously | continuous hydrogels |
| | distributed in another, | are composed of |
| | continuous hydrogel | different |
| | phase | functionalised triblock |
| | | molecule-silica |
| | | hybrid/composites and |
| | | aqueous liquid |
| Two-phase | Dispersed hydrogel | Dispersed hydrogel is |
| hybrid/composite # 2 | particles homogenously | composed of |
| | distributed in another, | functionalised triblock |
| | continuous hydrogel | molecule and aqueous |
| | phase | liquid, and the |
| | | continuous hydrogel of |
| | | silica and aqueous |
| | | liquid |

According to an embodiment, the functionalised triblock molecule of formula (1) and silica form colloidal particles, and a network of said particles forms a continuous solid phase, in which the aqueous liquid phase is homogeneously distributed, and the said solid and aqueous liquid phase are in a single hydrogel entity. This is the continuous hybrid/composite material of Table 1.

In this embodiment, the hydrogel is thus a homogeneous mixture of solid particle aggregates (colloidal aggregates formed from colloidal particles) and aqueous liquid. The silica is at least partly in the form of colloidal particles and colloidal aggregates, and forms a chemical bond with the triblock molecule, making it a hybrid material. The colloidal silica particles are formed from spherical clusters of molecules, and the atoms on their surfaces can form chemical bonds with other compounds in the material, including the triblock molecule but also other colloidal silica particles.

The functionalised triblock molecule and silica are both structures in the nanoscale. The functionalised triblock molecule is a molecule soluble or partially soluble in the aqueous liquid, and the silica is in the form of nanoparticles. The hybrid/composite if formed when the Si—OH— groups of the silica form a —Si—O—Si— bond with the Si—O— groups at the ends of the functionalised triblock molecule. One functionalised triblock molecule can also connect to another functionalised triblock molecule using these bonds, thus forming a network. Typically, each —Si—(O)₃— group may be bonded to 0-3 functionalised triblock molecules or 0-3 silica molecules.

Furthermore, the silica nanoparticles can also bond (or aggregate or agglomerate due to weak interactions) with one another. The silica nanoparticles may also form larger aggregates, but at least some of the nanoparticles are bonded to at least one functionalised triblock molecule.

Silica in these hydrogel materials is in the form of colloids or nanoparticles or aggregated nanoparticles (which is also a colloid). The four oxygens of silica bond to another molecule, for example to another silicon. Silica has a tendency to form a cyclic structure, and a cyclic (i.e. spherical) silica molecule quite quickly grows to be of such size that it is no longer soluble in the surrounding liquid, thus it can be called a nanoparticle or a colloid. Indeed, it forms its own solid phase in the surrounding liquid, but it is small enough to be able to stay stable in the liquid, and to move around due to Brownian motion in the same manner as soluble molecules. The surfaces of the particles have a number of free Si—OH-groups, which then react with the functionalised triblock molecules to form a chemical bond, thus forming a hybrid/composite.

In this first type of material, the nanostructures, i.e. functionalised triblock molecules and silica nanoparticles aggregate (or polymerise) and form a non-flowable hydrogel with the aqueous liquid, once the aggregation is sufficiently advanced. In case the hydrogel material is used to encapsulate another molecule, this molecule is added to the mixture before the non-flowable hydrogel is formed.

According to another embodiment, the hydrogel material further comprises a second hydrogel comprising
0.15-0.8 wt-% of the functionalised triblock molecule of formula (1), based on the total weight of the second hydrogel,
0.85-3.3 wt-% silica, based on the total weight of the second hydrogel; and
75-99 wt-% of an aqueous liquid, based on the total weight of the second hydrogel,
   wherein the —Si—OH groups of the silica form a —Si—O—Si— bond with the —Si—(O)₃— group of the functionalised triblock molecule of formula (1) of the second hydrogel, and
   wherein the second hydrogel is in the form of homogeneously distributed particles dispersed within the first hydrogel, the first hydrogel forming a continuous phase, and provided that the total amount of silica and functionalised triblock molecule having a formula (1) is higher in the second hydrogel than the total amount of silica and functionalised triblock molecule having a formula (1) in the first hydrogel.

This hydrogel material is the two-phase hybrid/composite #1 of Table 1. This hydrogel material thus has two different hydrogels, which are composed of the same constituents but in different amounts. The first hydrogel forms the continuous phase of the hybrid/composite #1. The second hydrogel has firstly been formed into a non-flowable hydrogel, and thereafter the physical structure of the second hydrogel has been broken up mechanically, to form particles. These particles are then mixed to the continuous phase of the first hydrogel, while the first hydrogel is still flowable. Typically, the second hydrogel (i.e. the dispersed phase) comprises less aqueous liquid than the first hydrogel.

According to an embodiment, the total amount of silica and functionalised triblock molecule having a formula (1) is at least 20% higher in the second hydrogel than the total amount of silica and functionalised triblock molecule having a formula (1) in the first hydrogel.

In case a biologically active agent or similar is to be encapsulated within this hybrid/composite #1, it is mixed with either the second hydrogel when it is still flowable, or to the first hydrogel, when it is still flowable, or to both. In case a slower delivery is aimed at, the agent is mixed with the second hydrogel, which is in particles in the final product. This hydrogel material is thus especially suitable when two different release profiles are aimed at, as it gives the possibility to deliver agents at two different speeds, depending on their location in the components of the hydrogel material.

According to yet another embodiment, in the hydrogel
the functionalised triblock molecule of formula (1) and a first part of the aqueous liquid are in the form of particles obtained by breaking a hydrogel of the functionalised triblock molecule of formula (1) and the aqueous liquid into particles, dispersed within a silica sol obtained by mixing a second part of the aqueous liquid; and
the —Si—OH groups of the silica have formed —Si—O—Si— bonds with the —Si—(O)₃— group of the functionalised triblock molecule of formula (1), thus forming the hydrogel material.

This hydrogel material is the two-phase hybrid/composite #2 of Table 1. The hydrogel material has two different parts, although having the same chemical components as the above-described hydrogel materials. In this embodiment, the continuous phase is formed by silica and aqueous liquid, and the dispersed phase is formed by first mixing functionalised triblock molecules with the aqueous liquid, allowing it to form a non-flowable hydrogel and the obtained hydrogel is mechanically broken up to particles. These particles are then mixed to the continuous phase, and the result is a hydrogel material. In case a biologically active agent or similar is added to the hydrogel, the addition is made by adding the agent to the mixture of functionalised triblock molecules and aqueous liquid while it is still flowable. This type of hybrid/composite #2 may be made injectable, as the silica gel forms the continuous phase.

In this embodiment, the final hydrogel material is formed in the same manner as the first hydrogel, i.e. chemical reactions lead to colloids, the colloids aggregate and the chemical bonds are of the same type in the first hydrogel and the final hydrogel material.

In the first and second hydrogel, the components are the same, i.e. the functionalised triblock molecule of formula (1), silica and aqueous liquid. The following amounts apply mutatis mutandis to the first hydrogel and the second hydrogel.

The amount of the functionalised triblock molecule of formula (1) is 0.15-21.0 wt-%, based on the total weight of the hydrogel. The amount of the functionalised triblock molecule of formula (1) can thus be for example from 0.15, 0.2, 0.25, 0.3, 0.35, 0.4, 0.45, 0.5, 0.55, 0.6, 0.65, 0.7, 1.0, 1.5, 2.0, 2.5, 3.0, 3.5, 4.0, 4.5, 5.0, 5.5, 6.0, 6.5, 7.0, 7.5, 8.0, 8.5, 9.0, 9.5, 10.0, 15.0, 18.5 or 19.5 wt-% up to 0.25, 0.3, 0.35, 0.4, 0.45, 0.5, 0.55, 0.6, 0.65, 0.7, 0.75, 0.8, 1.0, 1.5, 2.0, 2.5, 3.0, 3.5, 4.0, 4.5, 5.0, 5.5, 6.0, 6.5, 7.0, 7.5, 8.0, 8.5, 9.0, 9.5, 10.0, 15.0, 18.0, 19.5 or 21.0 wt-%, based on the total weight of the hydrogel.

The amount of silica is 0.85-4.0 wt-%, based on the total weight of the hydrogel. The amount of silica can thus be for example from 0.85, 1.0, 1.2, 1.5, 1.8, 2.0, 2.2, 2.5, 2.7, 2.9, 3.0, 3.1, 3.2, 3.3., 3.4, 3.5, 3.6 or 3.7 wt-% up to 1.0, 1.5, 1.8, 2.0, 2.2, 2.5, 2.7, 2.9, 3.0, 3.2, 3.3, 3.4, 3.5, 3.6, 3.7, 3.8, 3.9 or 4.0 wt-%, based on the total weight of the hydrogel

The amount of aqueous liquid is 75-99 wt-%, based on the total weight of the hydrogel. The amount of aqueous liquid can thus be for example from 75, 78, 80, 82, 85, 88, 90, 92 or 95 wt-% up to 80, 82, 85, 88, 90, 92, 94, 94, 95, 96, 97, 98 or 99 wt-%, based on the total weight of the hydrogel.

In the embodiments where one of the hydrogels are in the form of particles, the particle size of the second hydrogel or of the first hydrogel is less than 1 mm. Indeed, most typically the particle size would be a few tens of micrometres, so as to keep the material flowable through a needle of for example 25-27 G. Preferably, the particle size is less than 0.3 mm.

The particle size can thus be for example from 1, 5, 10, 15, 20, 25, 30, 35, 40, 45, 50, 55, 60, 65, 70, 75, 80, 85, 90, 95, 100, 120, 150, 200, 250, 300, 350, 300, 350, 500, 550, 600, 650, 700, 750, 800 or 850 μm up to 10, 15, 20, 25, 30, 35, 40, 45, 50, 55, 60, 65, 70, 75, 80, 85, 90, 95, 100, 120, 150, 200, 250, 300, 350, 300, 350, 500, 550, 600, 650, 700, 750, 800, 850, 900, 950 or 1000 μm.

In the present hydrogel material, the silica is preferably an alkoxysilane-derive silica, more preferably tetraethoxysilane-derived silica.

The aqueous liquid of the first hydrogel and of the second hydrogel is preferably independently selected from water; a mixture of water and ethanol; and biologically compatible buffers. Examples of such biologically compatible buffers are phosphate-buffered saline (PBS), citrate, succinate, acetate, benzoate and mixtures thereof.

According to a preferred embodiment, the aqueous liquid is a mixture of water and ethanol, comprising 50-95 wt-% of water, the rest being ethanol. The mixture may thus comprise for example from 60 wt-% up to 93 wt-% of water, the rest being ethanol. The amount of water can thus be from 50, 55, 60, 65, 70, 75, 80 or 85 wt-% up to 60, 65, 70, 75, 85, 90 or 95 wt-% of the total weight of the aqueous liquid. The aqueous liquid may also be solely water.

The present hydrogel material may further comprise at least one biologically active agent encapsulated therein. The biologically active agent may be encapsulated in different parts of the hydrogel, depending on its structure and intended use of the hydrogel material. Indeed, the biologically active agent may be encapsulated within the first hydrogel, within at least one of the first hydrogel and the second hydrogel and/or within the gel of the functionalised triblock molecule of formula (1).

According to an embodiment, the biologically active agent is selected from a group consisting of immunomodulatory agents and therapeutically active agents.

The encapsulation of the biologically active agent may be for various uses, such as delivery (for example oral or parenteral delivery), administration, desensitisation or for protection of the biologically active agent (for example against heat).

The biologically active agent may thus be for example immunomodulatory agents, such as vaccines and desensitisation agents for allergies. Some examples are antigens, viruses, specific antigens and virus-like particles (VLPs). Some examples of therapeutically active agents, also called drugs, are drug molecules of different sizes, such as peptides, proteins, biological drugs, biopharmaceutical drugs, biosimilar drugs, biobetter drugs, nucleic acid based drugs, cells and viral vectors. With vaccines, it has been observed in an in vivo test, that the antigen could be administered without any additional adjuvant (as usually used with vaccines), when the antigen was encapsulated in the present hydrogel material.

The present description also relates to use of a hydrogel material as explained above, for controlled delivery of a biologically active agent. It also relates to use of such hydrogel material for protective encapsulation of a biologically active agent. The protective encapsulation may be for example to ensure the stability of the biologically active agent and/or for its protection until controlled release.

In one particular embodiment, the protective encapsulation is to ensure the thermal stability of the biologically active agent. By thermal stability it is meant that the encapsulation ensures that the biologically active agent is not destroyed or denatured or altered in any way, if a product containing it is subjected to temperatures above typical refrigeration storage temperature (4-8° C.). It may also protect the biologically active agent during long-term or short-term storage at ambient temperature (20-25° C.) or at elevated temperature (>25° C., for example at 35-50° C.).

Furthermore, the present hydrogel may be used for causing adjuvant effect in immunisation using a biologically active agent, which means that the hydrogel delivered together with the antigen causes enhanced immune response as compared to administration of the antigen alone.

The present hydrogel material can be manufactured with various methods.

One possible method for manufacturing the functionalised triblock molecule comprises steps of
reacting L-lactide and polyethylene glycol having a molecular weight of 200-30000 g/mol, in a molar ratio of polyethylene glycol to L-lactide of 2:1-20:1, in the presence of a first catalyst, to obtain triblock molecules; and
reacting the obtained triblock molecules with isocyanate propyl triethoxysilane in the presence of a second catalyst, in a molar ratio of triblock molecule to isocyanate propyl triethoxysilane of 1:2, to obtain functionalised triblock molecules.

The second hydrogel is prepared in the same manner as the first hydrogel above, with the exception that the amounts used are different, in order to obtain a second hydrogel having the same constituents but in different relative amounts. In both cases, the aim is to have an equal number of L-lactide groups on both end-groups of the polyethylene glycol molecules, thus the molar ratio being any even number within the above-mentioned range.

The silica sol may be prepared for example by hydrolysing tetraethyl orthosilicate (TEOS) at pH 2 under stirring. The molar water-to-TEOS ratio (R) can be for example 98 (R98). After hydrolysis, the silica sol is let to age at room temperature, for example for 115 minutes, after which the pH is adjusted to pH 7.2-7.4 using 0.1 M NaOH.

TEOS is partially transformed to ethanol during the reaction. When the amount of water is even more in excess compared to TEOS (for example R200-R400), the resulting hydrogel materials are more fluid and can typically be injected using a thin needle. It is to be noted that the obtained silica sol does not contain any TEOS.

The single-phase hydrogel material can be prepared by dissolving the functionalised triblock molecule in an aqueous solution, and adding this mixture into the silica sol. Thereafter, the mixture is allowed to age to form the hydrogel material.

The two-phase hybrid/composite hydrogel material #1 (as explained above in Table 1) may be prepared by dispersing the second hydrogel material into gel particles by introducing shear forces by an injection through a thin needle.

For preparing the two-phase hybrid/composite hydrogel material #2, the hydrogel material comprising the functionalised triblock molecules is allowed to form a non-flowing hydrogel, and this hydrogel is then broken down into particles. The particles are added to a freshly made silica sol with mixing.

Other options for forming the hydrogel into particles comprise the following:
applying pressure on the gel, for example by pressing it; this leads to a paste-like product
applying lateral shear forces on the gel; this leads to a paste-like product or even a liquid product
mixing the gel, which is in essence similar to applying lateral shear forces; the use of low rotation speeds leads to a paste-like product, and the use of high rotation speeds leads to fluidic gel
applying vibration to the gel, for example vortexing; the original homogenous gel breaks down to smaller pieces, and the formed pieces are paste-like
applying a fast decomposition by using Turrax-mixing; the gel breaks down into smaller components but when the components come into contact with the walls of the recipient, the gel returns to paste-like
cutting the gel with a sharp object; practically no paste-like gel is formed, the volume is increased and the material becomes statically charged
shredding the gel; no paste-like gel is formed, the volume is increased and the material becomes statically charged
breaking up the gel by pushing it through a sieve (the gel needs to be sufficiently aged, for example 3 days or more); this leads to a porous and fluffy gel, the volume may increase to four-fold, and the material becomes statically charged
applying a mechanical crushing to the gel, for example by mortar and pestle, or by a ball mill or other types of mills.

### DETAILED DESCRIPTION OF THE DRAWINGS

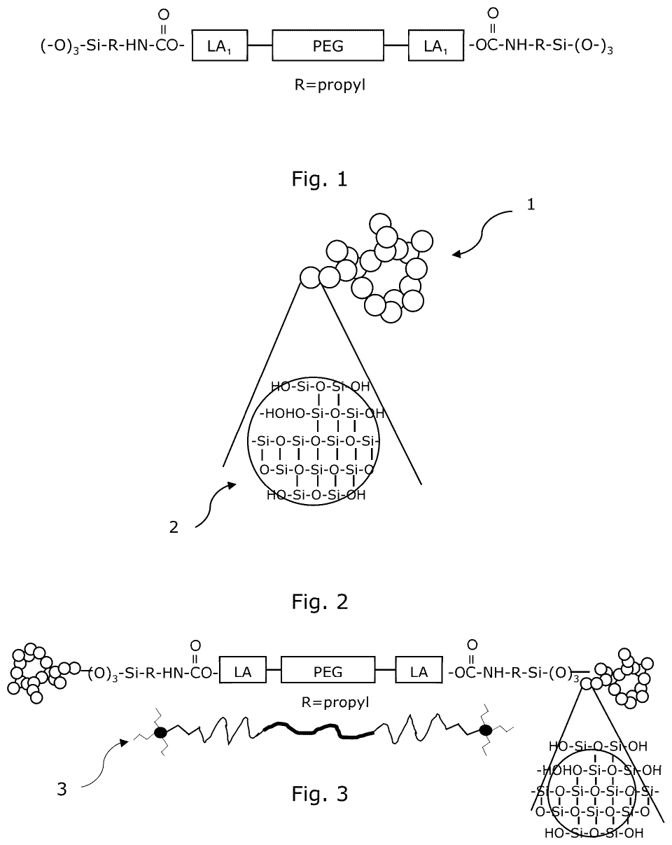

FIGS. 1 to 5A schematically illustrate the structures of various intermediate products and of the final hydrogel material. They are not to be construed as being on scale or exact reproductions of the structure, as the exact three-dimensional structure at nanoscale is not precisely known.

FIG. 1 schematically illustrates the structure of a functionalised triblock molecule. In this Figure, LA₁ stands for lactide and PEG stands for polyethylene glycol. R is propyl in this Figure.

FIG. 2 schematically illustrates silica nanoparticle aggregates in a silica sol. The silica sol comprises various structures 1, i.e. aggregates made of silica particles, of which one is shown as the enlargement 2.

FIG. 3 schematically shows a structure of a reaction product between silica and the functionalised triblock molecule, according to an embodiment. It can thus be seen that the silica particles shown in FIG. 2 are bonded at the ends of the functionalised triblock molecule. The schematic drawing shown with reference number 3 illustrates the relative sizes of the various parts of the product.

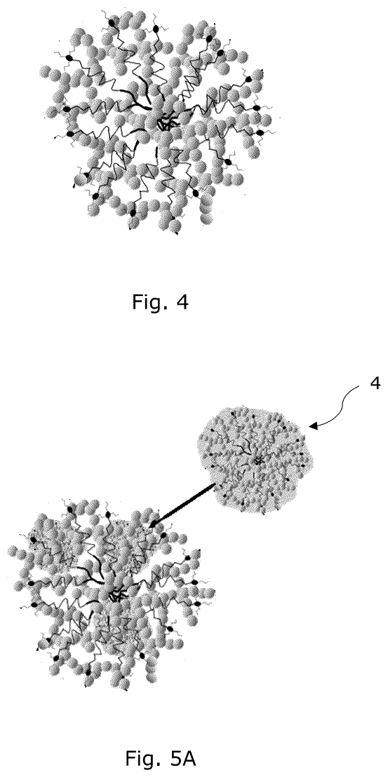

FIG. 4 illustrates a structure of the hydrogel material, according to an embodiment. As can be seen, the hydrogel material forms a network structure.

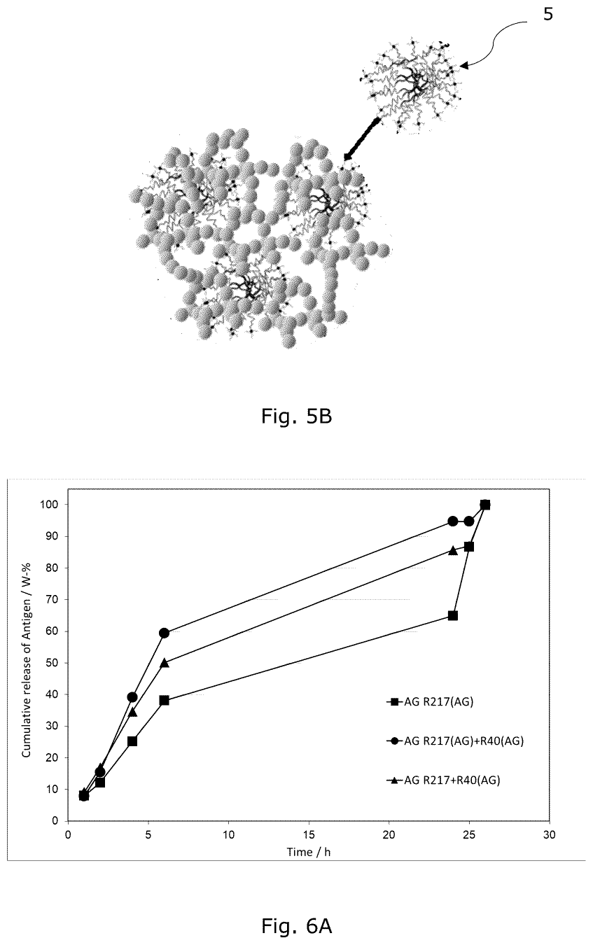

FIG. 5A illustrates a structure of the hydrogel material, according to another embodiment, namely the two-phase hybrid/composite hydrogel #1 as described above. The enlargement 4 shows the sub-structure of the hydrogel #1. FIG. 5B illustrates a structure of the hydrogel material, according to yet another embodiment, namely the two-phase hybrid/composite hydrogel #2 as described above, and the enlargement 5 shows the sub-structure of the hydrogel #2.

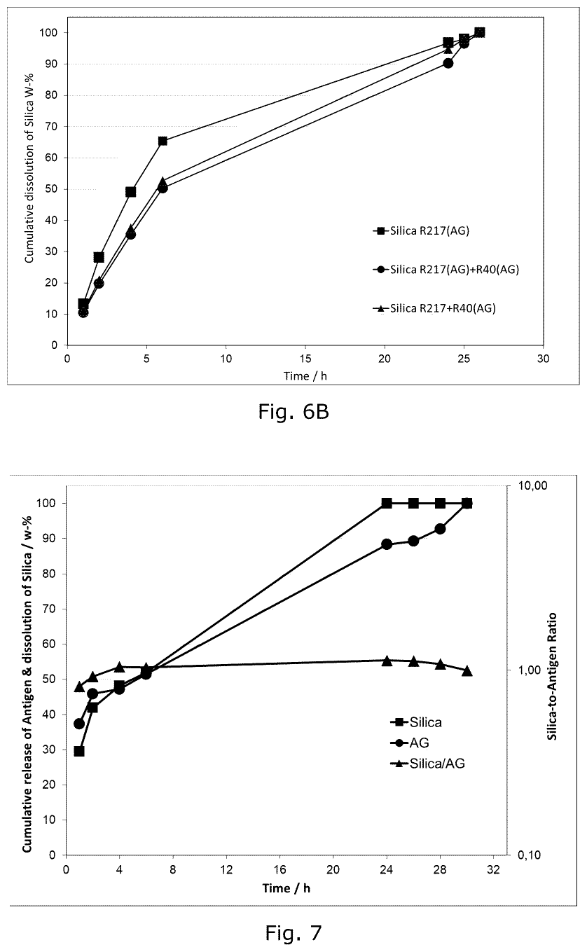

FIGS. 6 to 27B illustrate results of the Experiments, which are discussed in more detail in the Experimental part.

### EXPERIMENTAL PART

### Materials and Methods

### Preparation of Hydrogels

Three different types of hydrogel materials were prepared, as described above in Table 1.

The preparation of the hydrogel material consists of several steps. The first step is the preparation of the functionalised triblock molecule by first preparing a linker molecule, followed by preparation of a primary triblock, and functionalisation of the triblock silica, here with isocyanate propyl triethoxysilane. This functionalised triblock molecule is as shown with formula (1) and FIG. 1.

On the other hand, a silica sol is preparation of silica sol, as illustrated in FIG. 2. Thereafter, a hybrid/composite is prepared from the of silica sol and the functionalised triblock, illustrated in FIG. 3, and the hybrid/composite hydrogel is allowed to form (shown in FIG. 4).

The two-phase hybrid/composite hydrogels #1 and #2 were prepared by dispersing the other hydrogel into particles followed by mixing of the dispersed hydrogel particles into another hydrogel that worked as a continuous phase, illustrated in FIGS. 5A and 5B.

### Preparation of Continuous Hybrid/Composite Hydrogels and Norovirus P-Particle/Protein Embedment

The formation of triblock was initiated by carrying out a ring-opening polymerisation (ROP)-like reaction of L-lactide (Sigma Aldrich) to polyethylene glycol (PEG, Sigma Aldrich) (which also acts as the initiator) catalysed by Tin (II) 2-ethylhexanoate to prepare a linker molecule (both ends of PEG end-capped by L-lactide). The operation was conducted as bulk polymerisation without a solvent within an inert atmosphere Schlenk line system under N₂. The required amount of middle block (polyethylene glycol, PEG, 200 g/mol) was calculated according to molar ratio 2:1 (PLA: PEG). PEG was added, after which the temperature was raised to 160° C. under gentle stirring. The mixture was heated until the contents melted. Tin (II) 2-ethylhexanoate (Sigma Aldrich) in molar ratio 0.05 (catalyst:PEG) was pipetted into the melt and the vessel was sealed. The reaction was left to proceed for 90 minutes at 160° C. After the reaction, the product was cooled down to ambient temperature. The product was purified in a two-solvent system with chloroform and hexane (Sigma Aldrich). The hexane supernatant was decanted, and the chloroform was evaporated in two steps. First, the chloroform was evaporated in a rotary evaporator (IKA RV10) at 450 mbar vacuum at 40° C. for 15 minutes. In the second step the last traces of solvents were evaporated with a vacuum pump. The obtained triblock was warmed to +60° C. in a water bath and pressure was lowered to p <0.1 mbar by a diffusion pump (Vacuubrand RZ 2.5). Any evaporating substances were captured in a solvent trap immersed in liquid N₂. This procedure was carried on until no more bubbles were forming on the melt.

The prepared triblock was then reacted with isocyanate propyl triethoxysilane (IPTS, Sigma Aldrich) to produce a functionalised triblock molecule. This was done by dissolving the triblock, IPTS and dibutyltin dilaureate (catalyst, Alfa Aesar) in tetrahydrofurane (THF, Sigma Aldrich) at molar ratios 1:2:0.05. The mixture was heated to 60° C. under inert atmosphere using Schlenk line system as described in the triblock preparation step above. The reaction was left to proceed for 1 hour. THF was evaporated with a rotary evaporator (IKA RV10) at 357 mbar vacuum at 40° C. for 10 minutes. The obtained functionalised triblock molecule was then then dissolved in a two-solvent system of hexane and chloroform. Further purification steps of the functionalised triblock molecules were carried exactly out as described above in the triblock preparation section.

Next, a typical example of the other steps in the hybrid/composite hydrogel preparation process is described. Several different formulations were prepared, where polymer molecular weight (i.e. the molecular weight of the polyethylene glycol), and the amounts of P-particle, functionalised triblock molecules, water and silica were varied, but the preparation process was otherwise the same, only mixing, aging and gelation times varied slightly.

The next step after the synthesis of the functionalised triblock molecule was preparation of the silica sol. Tetraethyl orthosilicate (TEOS, Sigma Aldrich) was used as silica precursor. TEOS was hydrolysed at pH 2 (adjusted using 0.1 M HCl, Merck Titripur) under stirring. The molar water-to-TEOS ratio (R) was 98 (R98). After hydrolysis, the silica sol was let to age at room temperature for 115 minutes, after which the pH was adjusted to pH 7.2-7.4 using 0.1 M NaOH (Merck Titripur). The resulting silica sols were filtered with sterile 0.45 μm PES membrane syringe filters.

200 mg of functionalised triblock molecule (with PEG 200 g/mol) was dissolved in 50 ml in phosphate-buffered saline (PBS, Sigma Aldrich) containing norovirus P-particles with the concentration of 200 micrograms/ml (200 ppm). This mixture was then added into 50 ml of R98 silica sol (aged at pH 7.2-7.4 about 3 minutes) yielding to P-particle concentration of 100 ppm in the resulting sol. The sol was transferred into syringes (of 1 ml) within 2-5 minutes before the sol (total volume about 100 ml) turned into a non-flowing gel (gelation time varies depending on the formulation details). Evaporation of the aqueous solution (water and ethanol) was minimised during the preparation and pH adjustment steps by covering the vessels with a plastic film. The resulting water-to-TEOS ratio (R) is 217 (R217) corresponded to 1.5 wt-% of silica in the final hydrogel material. The final concentration of functionalised triblock molecule was 0.2 wt-%, and P-particle concentration 100 ppm, i.e., 0.1 mg in 1 ml of the final hydrogel material. In the example above, the final coding for the hydrogel material is R217-0.2, indicating both silica and functionalised triblock molecule content of the final material. The composition of R217-0.2 hydrogel is provided in detail in Table 2.

TABLE 2

| | |
|---|---|
| Wt-% of functionalised | 0.20 |
| triblock molecule | |
| Wt-% of silica | 1.49 |
| Wt-% of water | 93.26 |
| Wt-% of ethanol | 4.56 |
| Wy-% of antigen | 0.01 |
| Wt-% of residuals | 0.48 |
| (NaCl) | |

The actual hydrogels consisted of functionalised triblock molecules, silica, water and ethanol, in which the antigen was encapsulated. In addition to that, hydrogels may contain some encapsulated residuals, i.e., NaCl in the structure. Different corresponding hydrogel materials with different component concentrations were prepared (R217-0.15, R328-0.2, R328-0.4, R328-0.8, R40-0.2). Higher molar masses for PEG (2000 g/mol and 6000 g/mol) were also tested for continuous hybrids/composites with R217, but their injection properties were not optimal, and thus more extended studies on hybrids/composites were conducted only with PEG 200 g/mol in the triblock. However, such materials (i.e. with higher molar masses for PEG) are useful for delivery purposes for non-injectable applications, as their properties are otherwise similar to those prepared and tested below.

Preparation of two-Phase Hybrid/Composite Hydrogels and Norovirus P-Particle/Protein Embedment

The functionalised triblock molecule described above is a part of both two-phase hybrid/composite hydrogels #1 and #2 (as listed above in Table 1). In two-phase hybrid/composite hydrogel #1 pieces of dispersed hydrogels are composed of same solid phase as in the continuous phase, but the amount of water is different. In two-phase hybrid/composite hydrogel #2 the continuous hydrogel is an inorganic silica gel and the dispersed component is an organic hydrogel. The general structures of the two-phase hydrogels are illustrated in FIGS. 5A and 5B. The actual hydrogels consist of functionalised triblock molecules, silica, water and ethanol, in which the antigen is encapsulated. In addition to that hydrogels may contain some encapsulated residuals, i.e., NaCl in the structure.

In two-phase hybrid/composite hydrogel #1 (Table 3) both components are of same type, comprising an alkoxysilane-functionalised triblock molecule-silica hybrid/composite (organic-inorganic hybrid/composite), the only difference is the amount of water used in the hydrogels. Different two-phase hydrogels with different molar water-to-TEOS ratio (R40, R217 and R328) and functionalised triblock molecule content (0.15 and 0.2 wt-% for R217, 0.2, 0.4 and 0.8 wt-% for R328 and 0.2 wt-% for R40) were prepared, but R217-0.2/R40-0.2 was selected for more closer characterisation due its good rheological properties and injectability. The former component (here R217-0.2) indicates the continuous hydrogel phase, and the latter component (here R40-0.2) is the hydrogel that is dispersed into the continuous hydrogel phase. Norovirus P-particles were embedded in a corresponding way as described above either only in the dispersed hydrogel, or both in the dispersed and continuous hydrogel. In addition, monomeric enhanced green fluorescent protein (eGFP) was used as another encapsulated agent in R217-0.2. The dispersed hydrogel R40-0.2 was prepared by letting the system form a non-flowing gel first (ensures effective embedment of P-particles). Right after the hydrogel formation, the non-flowing R40-02 was dispersed into gel particles by introducing shear force by thin needle (18 G) injection. This step was carried out rapidly (within 1-2 minutes) in order to avoid that the aqueous liquid (water and ethanol) evaporates from the system. The main function of the continuous hydrogel is to act as an injection matrix, i.e., it provides suitable rheological properties for thin needle injections for the two-phase system, but it can also be used as a part of the controlled release system, e.g., in order to achieve different release rates, e.g., two-phase release.

Table 3 gives the composition of two-phase hybrid/composite hydrogel #1 (R217-0.2/R40-0.2) with encapsulated antigen (A: antigen in both R217 and R40 with mass ratio 25:75, and B: antigen only in R40) and residuals.

TABLE 3

| Component, wt-% | R217-0.2/R40-0.2 A | R217-0.2/R40-0.2 B |
|---|---|---|
| Functionalised | 0.20 (0.10 + 0.10) | 0.20 (0.10 + 0.10) |
| triblock molecule | | |
| Silica | 3.25 (0.74 + 2.51) | 3.25 (0.74 + 2.51) |
| Water | 86.01 (46.59 + 39.42) | 86.01 (46.59 + 39.42) |
| Ethanol | 10.05 (2.28 + 7.77) | 10.05 (2.28 + 7.77) |
| Antigen | 0.02 (0.005 + 0.015) | 0.02 (0 + 0.02) |
| Residuals (NaCl) | 0.47 (0.236 + 0.234) | 0.47 (0.236 + 0.234) |

In two-phase hybrid/composite hydrogel #2 the continuous component was a silica hydrogel and the dispersed component a hydrogel composed of alkoxysilane-functionalised triblock molecules and water. Four different formulations of hydrogels with different weight ratios between silica, siloxane-functionalised functionalised triblock molecules and water, were prepared (Table 4). Tetraethyl orthosilicate (TEOS, Sigma Aldrich) was used as silica precursor. TEOS was hydrolysed at pH 2 (adjusted using 0.1 M HCl) under stirring. The molar water-to-TEOS ratio (R) was 220 (R220) for each formulation. Additional ethanol was used as in the aqueous solution in order to establish a homogeneous system with silica, functionalised triblock molecules and water (formation of silica sol from TEOS results in formation of by-product, ethanol). The pH of the resulting silica was adjusted to pH 5-6 (using 0.1 M NaOH) prior to addition of pieces of organic hydrogel comprising the siloxane-functionalised triblock molecule and water. Dispersed organic hydrogel preparation was started by letting the system form a non-flowing hydrogel first (ensures effective embedment of P-particles). After hydrogel formation, the structure was dispersed into particles by grinding the hydrogel into hydrogel particles. Addition of the dispersed organic hydrogel particles into the freshly made silica sol were conducted quickly in 1-2 minutes in order to avoid separation of water and the whole system was homogenised using Ultra-Turrax T25 (with stator S25N-18 G). The method for dispersing the organic hydrogel was different than that for two-phase hybrid/composite hydrogel #1, because the solid content of different formulations of two-phase hybrid/composite hydrogel #2 is higher. The mixture of silica gel and particles of organic hydrogel turns into a non-flowing hydrogel in about 30 minutes, and prior to the hydrogel formation mixing is on in order to avoid the sedimentation of the organic hydrogel pieces.

Table 4 shows the different formulations of two-phase hybrid/composite hydrogel #2 with encapsulated antigen and residuals.

TABLE 4

| Component, wt-% | A | B | C | D |
|---|---|---|---|---|
| Functionalised | 8.33 | 12.50 | 16.66 | 20.83 |
| triblock molecules | | | | |
| Silica | 1.19 | 1.04 | 0.89 | 0.75 |
| Water | 81.03 | 74.54 | 68.07 | 61.56 |
| Additional | 5.83/3.58 | 8.75/3.13 | 11.66/2.68 | 14.58/2.24 |
| Ethanol/Ethanol | | | | |
| formed (silica sols) | | | | |
| Antigen | 0.01 | 0.01 | 0.01 | 0.01 |
| Residuals (NaCl) | 0.03 | 0.03 | 0.03 | 0.03 |

Norovirus P-particles were embedded in the two-phase hybrid/composite hydrogel #2 in as described above (i.e., the P-particle solution was added into the system while it still had a flowing, liquid-like structure, i.e., prior to the hydrogel formation), but in this case only in the organic hydrogel, which was dispersed. If desired, also here P-particles or other biologically active agents can be embedded both in the dispersed and continuous hydrogel.

eGFP Production and Properties

Enhanced green fluorescent protein (eGFP) was produced using genetically modified E. coli (a gene encoding for monomeric enhanced green fluorescent protein was transformed to E. coli) as a production organism. The protein was purified using standard methods in molecular biology.

To embed the eGFP protein into hydrogel, the following procedure was used. The protein stock used in the experiments was produced in 2017 and stored at −20° C. The protein content was measured with μBCA (a micro BCA Protein Assay Kit (Microplate Procedure, Thermo Scientific™) prior to the experiments described above. The concentration was reported to be 2.2 mg/ml. The molar mass of eGFP was determined with SDS-PAGE (sodium dodecyl sulphate polyacrylamide gel electrophoresis) to be approximately 40 kDa.

### Dissolution Experiments

Release of P-Particles and eGFP and Biodegradation of Hybrid/Composite Hydrogels

The dissolution experiments, i.e., release of P-particles and dissolution of the hybrid/composite hydrogels (R217-0.2 and R217-0.2-R40-0.2), were conducted either in PBS or in tris(hydroxymethyl)aminomethane (TRIS) buffered to about pH 7.4 at 37° C. in a shaking bath. PBS was used for the release of P-particles, and TRIS for the dissolution of the inorganic-organic hybrid/composite. The dissolution experiments were conducted in sink conditions (to ensure free dissolution without preventing the dissolution rate due to dissolution products) meaning in practice the in sink conditions for the inorganic part (amorphous silica) of the hybrid/composite hydrogel. The dissolution medium was regularly refreshed at every sampling time point to maintain the in sink conditions (<30 ppm for concentration of dissolved amorphous silica). Three replicates were collected at each time point.

The release of P-particles and eGFP was analysed with total protein analysis method, a micro BCA Protein Assay Kit (Microplate Procedure, Thermo Scientific™) for colorimetric detection at 562 nm by a spectrophotometer (Hidex Sense Microplate Reader). Dissolution rate of hybrid/composite hydrogels (continuous hybrid/composite and two-phase hybrid/composite #1) was estimated by measuring the dissolution of silica as function of time by GF-AAS (Shimadzu 6650F, GFA-EX7), which corresponded to 3.5% (of two-phase hybrid/composite hydrogel #2-D)-82.5% (of two-phase hybrid/composite hydrogel #1-B) of the total solid content hybrid/composite hydrogel. Prior to analysis, the liquid samples containing P-particles were concentrated. The concentration was conducted because the dissolution measurement were conducted in sink conditions (with respect to silica) that resulted in very diluted samples (and in sink conditions were used in order to find out the real mechanism of release taking into account both biodegradation- and diffusion-related component in the release, and in order to observe real difference between different material versions). The in sink limit of silica was about 30 ppm, and thus maximum concentration of any encapsulated substance would be related to the same limit according to loading percentage. If loading percentage with respect to the total mass of silica is 1%, the maximum concentration in a dissolution sample for the encapsulated substance is thus only 0.3 ppm. The concentration of the samples was conducted either by centrifugal membrane filtration (Eppendorf Centrifuge 5810 R at 8000-10000 G for 15-30 minutes with Amicon® Ultra-15 centrifugal filters Ultracel®-3K) or by lyophilisation (Christ Epsilon 1-6 D, load and maximum freezing temperature at −35° C. followed by gradual increase of temperature to -15° C. in main drying, and final drying at −10° C. for 23 min and at 20° C. for 20 min) followed by redispersion of the lyophilised P-particles into a smaller liquid volume.

Dissolution of P-Particles from Hydrogel 8217 for the Study of Particle Composition and Integrity

To study if the released the antigen was intact after being kept in different temperatures, hydrogels containing 0.5 mg/ml of P-particles were dissolved in 10 kDa cut-off dialysis cassettes (Slide-A-Lyzer, ThermoFisher). First, hydrogels were removed from syringes, weighted and transferred into dialysis cassettes. Cassettes were dialysed in 0.8 I of PBS that was changed five times over one week at room temperature.

Next, after checking that no gel remnants were present in cassettes, the solution from the cassettes was transferred to microcentrifuge tubes, aliquoted and stored for analyses at −20° C.

### Release Mechanism Experiment

The release mechanism of P-particles was studied in a silica-saturated PBS or in Tris(hydroxymethyl)aminomethane (TRIS) buffered to about pH 7.4 at 37° C. in a shaking bath. The silica-saturated medium was prepared by dissolving a piece of amorphous sol-gel derived silica in the medium until the saturation level of silica (about 130 ppm) was reached. When dissolution of the major part (silica) of the hybrid/composite hydrogel is prevented, the P-particles can mainly be released by diffusion only. The diffusion results are then compared with the release experiments conducted in sink conditions, where both matrix biodegradation and diffusion may occur simultaneously. R217-based continuous hybrids/composites with encapsulated P-particles and eGFP were tested for the release mechanism.

### Rheological Measurement and Injection Experiments

Rheological measurements were carried out using a modular rheometer

(Anton Paar MCR 104). Samples were injected onto the geometry (20 mm plate-plate) from 1 ml syringe (Becton Dickinson) through a 25 G needle (Becton Dickinson Microlance). Amplitude sweep measurements were conducted at strain values 0.01-10% at a constant 6.28 rad/s angular frequency. Frequency sweeps were conducted at frequencies 0.01-100 rad/s at a constant strain defined by the amplitude sweep measurement.

Injectability of the prepared hybrid/composite hydrogel materials was investigated. Tests were performed for continuous hybrid/composite and two-phase hybrid/composite #1 hydrogels. Materials were injected with normal force from 1 ml syringes (Becton Dickinson) through a 25 G needle (Becton Dickinson Microlance). A line of hydrogel was injected on a plastic tray and the result was inspected visually. Smoothness of injections, signs of phase separation and apparent homogeneity and compatibility with the dissolution buffer were documented and evaluated.

### 2.4 Production and Purification of Norovirus P-particles

His-tagged norovirus P-particles were produced in E. coli BL21 star cells as described in Koho et al. (Journal of Virological Methods 179 (2012) 1-7). The pelleted bacterial cells were lysed with EmulsiFlex®-C3-homogenisator (Avestin Inc.) into lysis buffer (50 mM NaH₂PO₄, 600 mM NaCl, 10 mM imidazole, pH 8.0), after which the cell lysate was clarified at 10 000×g for 30 minutes at 4° C. and the clarified lysate was used for affinity purification using nickel-charged sepharose (Merck, HisTrap FF Crude). The purified P-particles were dialysed to PBS and thereafter they were sterile filtrated for the analyses and further use. The concentration of P-particles was measured with BCA assay (Pierce).

### Dynamic Light Scattering Analysis

Dynamic light scattering (DLS) analysis of P-particles was performed with a Zetasizer Nano ZS instrument (Malvern Instruments Ltd., Worcestershire, UK). The hydrodynamic diameter was determined using three 10×10-second datasets at 25° C. in PBS. The samples were also subjected to stepwise heating as described in Koho et al. (Antiviral Research 104 (2014) 93-101). In short, starting at 25° C., each sample was heated in 5° C. increments and equilibrated for 5 min at each temperature before analysis. The samples were heated to a final temperature of 90° C., after which they were cooled back to 25° C. To study the integrity and composition of P-particles dissolved from continuous hybrid/composite hydrogel R217-0.2 into PBS, they were analysed with DLS at 25° C.

### Endotoxin Determination

In order to confirm endotoxin removal from the purified antigen, endotoxin levels in P-particles were determined using ToxinSensor™ Gel Clot Endotoxin Assay Kit (GenScript) according to manufacturer's instructions.

### Production and Purification of Norovirus VLPs

Norovirus (NoV) GII capsid virus-like particles (VLPs) derived from GII.4 (1999, acc. no. AF080551), GII.4 New Orleans (NO; 2010, acc. no. GU445325), GII.4 Sydney (Syd; 2012, acc. no. AFV08795.1), GII.12 (1998, acc. no. A3277618), and GII.17 (2015, acc. no. BAR42289) were produced in Sf9 insect cells by Bac-to-Bac baculovirus expression system (Invitrogen, Carlsbad, CA) and purified by sucrose gradient ultracentrifugation as described in detail elsewhere (Huhti et al. Arch Virol 2010 155; Blazevic et al. Vaccine 2011 29; Maim et al. Clinical and vaccine immunology 2015 22). These VLPs were employed as antigens in vitro immunogenicity assays.

### Animal Immunisation

Pathogen-free female 6-week-old BALB/c OlaHsd mice (Envigo, Horst, the Netherlands) were randomly divided into eight groups (Gr I-VIII, 3 or 5 mice/experimental group), acclimatised under controlled specific conditions for a period of one week before starting the experiment. Animals were immunised twice with a 10 μg or once with a 20 μg dose of NoV P-particles diluted in sterile PBS (Lonza, Verviers, Belgium) or formulated with continuous hybrid/composite R217-0.2 or or two-phase hybrid/composite #1 R217-0.2/R40-0.2 (A and B, Table 3) hydrogels. Test articles were administered via subcutaneous (s.c.) injection into the right flank (100 μl volume) at study weeks 0 and 3. Table 5 shows the employed vaccine formulations, injection doses and immunisation regimens. Control groups received only R217-0.2 or R217-0.2/R40-0.2 hydrogel or P-particles formulated with Al(OH)₃ (Alhydrogel; InvivoGen, San Diego, CA). Immunisations were conducted under general anesthesia by inhalation of isoflurane (Attane vet, Vet Medic Animal Health Oy).

Blood samples were collected at study weeks 0 (pre-bleed, non-immune sera) and 3 by tail bleeding to test for the kinetics of the serum antibody responses. Whole blood and faeces were collected at the time of sacrifice (week 5 or 6) and processed according to the previously published procedures (“Norovirus VLPs and rotavirus VP6 protein as combined vaccine for childhood gastroenteritis” Blazevic et al, Vaccine October 19;29(45):8126-33.; “A comparison of immunogenicity of norovirus GII-4 virus-like particles and P-particles”, Tamminen et al., Immunology 2012 January;135(1):89-99). Experimental procedures were carried out in accordance with the regulations and guidelines of the Finnish National Experiment Board (Permission number ESAVI/10800/04.10.07/2016). All efforts were made to minimise animal suffering. Animal welfare was monitored throughout the study period.

Table 5 shows the antigenic formulations, injection doses and immunisation regimens.

TABLE 5
Ag stands for antigen, here P-particle

| | | | | Immunisa- | |
|---|---|---|---|---|---|
| | | Injection | Mice/ | tion | Termina- |
| Group | Test article | dose (μg) | group | schedule | tion |
| I | P-particle | 10 | 5 | w 0, w 3 | w 5 |
| II | P-particle + | 10 | 5 | w 0, w 3 | w 5 |
| | R217-0.2 | | | | |
| III | P-particle | 20 | 5 | w 0 | w 6 |
| IV | P-particle + | 20 | 5 | w 0 | w 6 |
| | R217-0.2/R40- | | | | |
| | 0.2 B | | | | |
| V | P-particle + | 20 | 5 | w 0 | w 6 |
| | R217-0.2/R40- | | | | |
| | 0.2 A | | | | |
| VI | P-particle + | 10 + 100 | 5 | w 0, w 3 | w 5 |
| | Al(OH)₃ | | | | |
| VII | −R217-0.2 | — | 3 | w 0, w 3 | w 5 |
| VIII | −R217-0.2/R40- | — | 3 | w 0 | w 6 |
| | 0.2 | | | | |

### Antigen-Specific Antibody Responses

Antibody responses generated against P-particles were determined measuring IgG and IgG subtype levels in serum samples of individual mice by ELISA. The procedural steps of the employed ELISA were similar to those previously published by our laboratory (Blazevic et al. Vaccine 2011 29; Tamminen et al. Immunology. 2012 135(1):89-99) and are only shortly outlined as follows. Half-area polystyrene plates (Corning Inc., Corning, NY) were coated with 50 ng of NoV P-particles per well. Antigen-specific antibodies in sera at 1:200 dilution or serially diluted two-fold were detected with a combination of HRP-conjugated anti-mouse IgG (Sigma-Aldrich), IgG1 (Invitrogen) or IgG2a (Invitrogen) and SIGMA FAST OPD substrate (Sigma-Aldrich). Endpoint titers were defined as the reciprocal of the highest sample dilution with an OD₄₉₀ above the cut-off value (>0.1 OD₄₉₀ unit).

### Antibody Avidity

The avidity of NoV GII.4 type-specific IgG antibodies was evaluated in 1:200 diluted sera according to ELISA method described above in 1.5. but accompanied with an additional urea treatment (Tamminen et al. Immunology. 2012 135(1):89-99) to eliminate the antibodies with low-avidity. The plates were coated with 50 ng of NoV GII.4 VLPs. Results were expressed as avidity index: (OD₄₉₀ with urea/OD₄₉₀ without urea)×100%.

### Cross-Reactive Antibodies

Cross-reactive NoV-specific IgG antibodies were detected with the ELISA as described above. but the plates were coated with 50 ng of heterologous NoV VLPs, including GII.4 NO, GII.4 Sydney, GII.12, and GII.17 VLPs, per well. Serum dilution of 1:200 was used in the assay.

### Blocking Antibodies

The capability of induced antibodies to prevent binding of NoV VLPs to HBGA receptors was assessed with blocking assay, employing PGM type III (Sigma Chemicals) as a HBGA source (Lindesmith et al. J Virol 2012; 86:873-83) according to the previously published procedure (Maim et al. Clin Exp Immunol 2017;189(3):331-41). Briefly, the mixtures of pre-incubated GII.4 VLPs and serially diluted sera were added on PGM-coated microwell plates. The bound VLPs were detected in combination of human NoV GII.4 antiserum and HRP-conjugated anti-human IgG according to the receptor binding assay described above in 1.2. Results were expressed as blocking index: 100%-[(0D490 sannple/OD490 max.binding)×100%].

### Mucosal IgG Response

For the detection of mucosal IgG antibodies, serially diluted 10% faecal suspensions were examined by the ELISA method described above in 1.5. The plates were coated with 50 ng of NoV GII.4 VLPs.

### Results and Discussion

In vitro Dissolution and Antigen Release

The in vitro dissolution results for antigen (P-particle) release rate and dissolution rate of silica for different hybrid/composite hydrogels are shown in FIGS. 6-8. The antigen (P-particle) release rate for the continuous hybrid/composite R217-0.2 and for two versions of two-phase hybrid/composite hydrogel #1; R217-0.2/R40-0.2 A (antigen (AG) both in R217-0.2 and R40-0.2 hydrogel) and R217-0.2/R40-0.2 B (antigen (AG) only in R40-0.2 hydrogel) are shown in FIG. 6A after storage in syringes enclosed in an aluminium foil bags for 4 and 330 days at room temperature (25° C.). FIG. 6B shows the cumulative release of silica in the same conditions.

FIG. 7 illustrates the cumulative release of antigen (P-particle) and dissolution of silica for two-phase hybrid/composite hydrogel #2C. Weight ratio between released amount of silica and P-particles at different time points is shown in secondary Y-axis on the right.

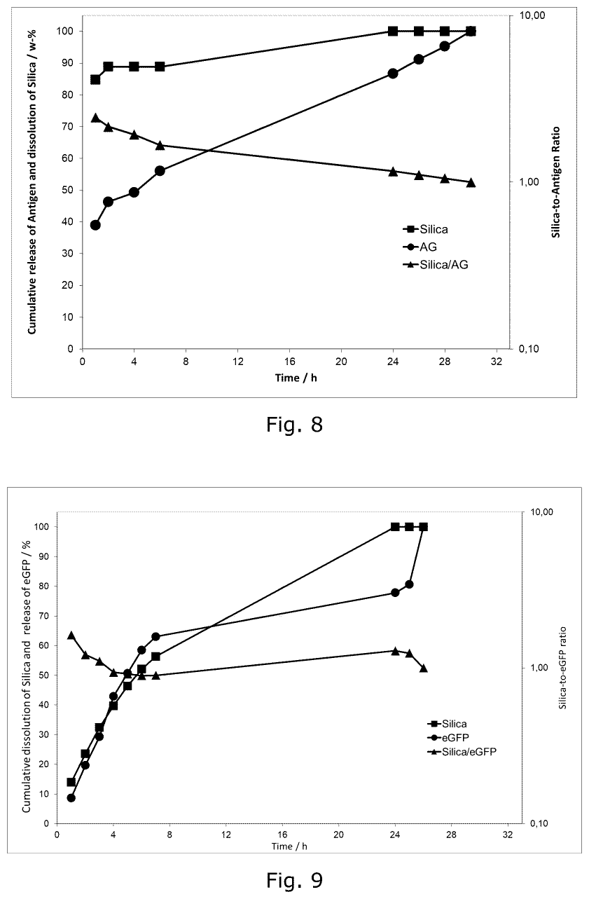

FIG. 8 illustrates the cumulative release of antigen (P-particle) and dissolution of silica for two-phase hybrid/composite hydrogel #2D. Weight ratio between the released amount of silica and P-particles at different time points is shown in secondary Y-axis on the right.

The release results for R217-0.2 with encapsulated eGFP show that dissolution of silica and release of eGFP occur quite simultaneously, but there is some difference with the encapsulated amount of eGFP. For 20 μg of eGFP in 100 μl of R217-0.2 both release rate of eGFP and dissolution rate of silica are a bit slower in the beginning, but they reach about 100% approximately at the same time.

FIG. 9 illustrates the cumulative release of eGFP and dissolution of silica for R217-0.2 with 10 μg of eGFP in 100 μl of hydrogel. Weight ratio between the released amount of silica and eGFP at different time points is shown in secondary Y-axis on the right.

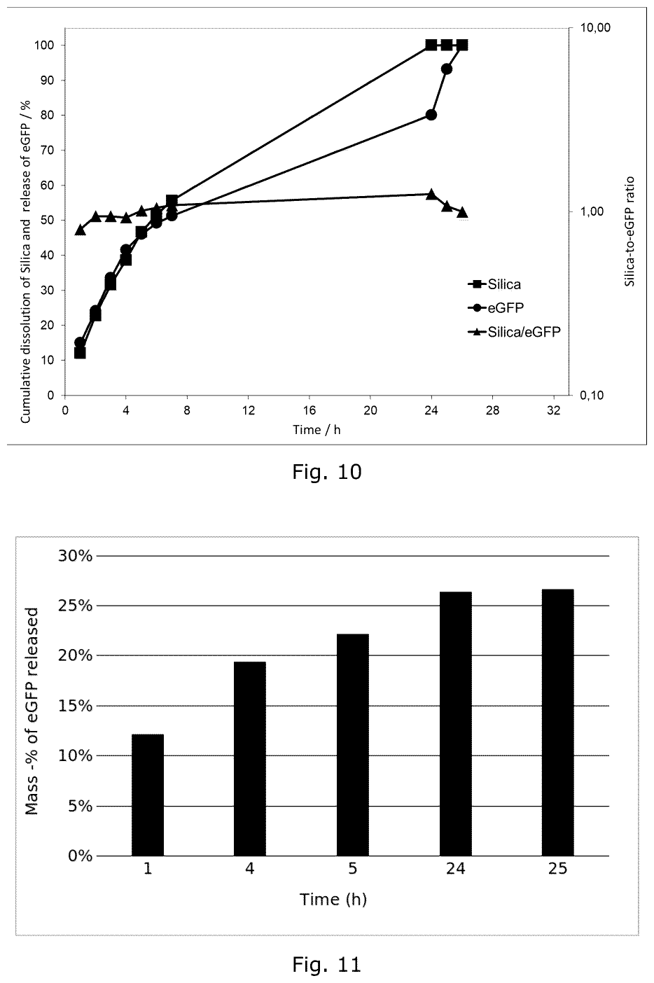

FIG. 10 illustrates the cumulative release of eGFP and dissolution of Silica for R217-0.2 with 20 μg of eGFP in 100 μl of hydrogel. Weight ratio between the released amount of silica and eGFP at different time points is shown in secondary Y-axis on the right.

The cumulative release of eGFP from silica-satured dissolution medium from R217-0.2 containing 10 μg and 20 μg of eGFP per 100 μl of hydrogel was measured to investigate the encapsulation efficiency of the material. The rate at which eGFP was being diffused out from the hydrogel during the first five hours was c.a. 50% slower than under in sink conditions, as shown in FIGS. 9 and 10. The release rate was slightly retarded after 24 hours, plateauing at 25% for 10 μg/100 μl material and at 35% for the 20 μg/100 μl version.

FIG. 11 shows the cumulative diffusion of eGFP from R217-0.2 with eGFP concentration of 10 μg/100 μl of hydrogel.

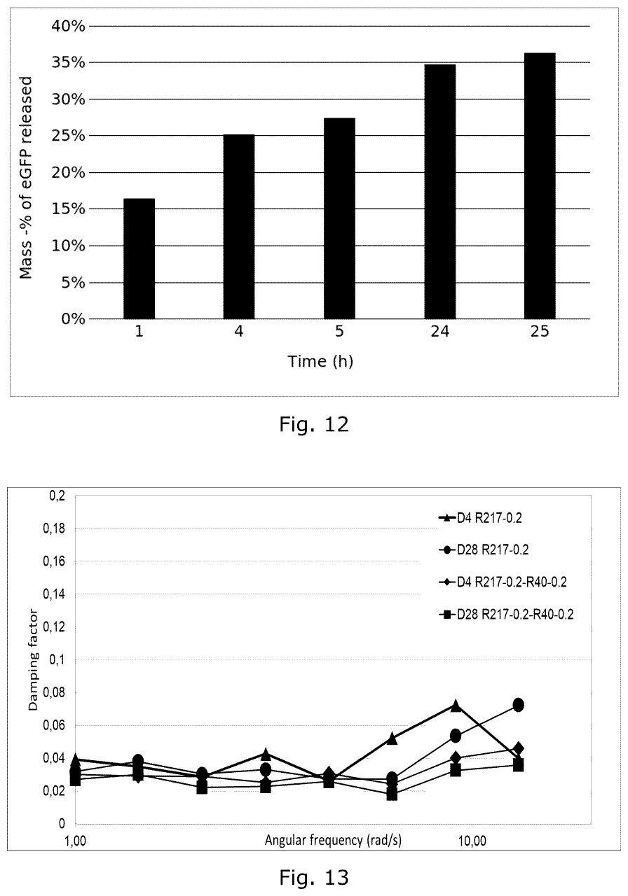

FIG. 12 illustrates the cumulative diffusion of eGFP from R217-0.2 with eGFP concentration of 20 μg/100 μl of hydrogel.

The concentration of eGFP at 24 or 25 hours is less than 0.5% of the stock concentration of the protein, and therefore it is unlikely that eGFP would hinder its own release in terms of saturation. Moreover, during the diffusion measurement, the sample volume in the case of 10 μg/100 μl, the release rate of eGFP reaches a plateau after 24 hours and remains somewhat stable, whereas, while similarities can be observed in 20 μg/100 μl version, it does seem to have yet stabilised. The comparison to the in sink dissolution suggests that while some diffusion does occur, the entire eGFP payload is not released as effectively without material degradation also taking place.

### Rheology

FIG. 13 shows the damping factor (G″/G′) for continuous hybrid/composite hydrogel R217-0.2 and for two-phase hybrid/composite hydrogel #1 (R217-0.2/R40-0.2) at day 4 and at day 28 after storage in syringes enclosed in an aluminium foil bags at room temperature (25° C.).

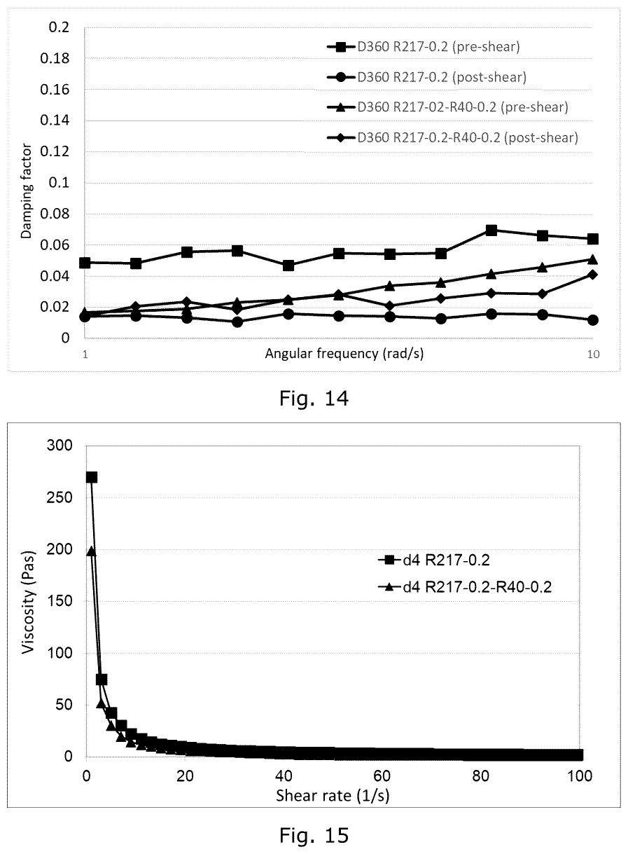

FIG. 14 shows the damping factor (G″/G′) for continuous hybrid/composite hydrogel R217-0.2 and for two-phase hybrid/composite hydrogel #1 (R217-0.2/R40-0.2) at day 360 after storage at room temperature (25° C.). Syringes were isolated in aluminium foil bags. The samples were subjected to a three-part measurement where the first step was a frequency sweep, followed by a rotational measurement (shear rate ramp 1-100 1/s) and again followed by a frequency sweep.

FIG. 15 illustrates the dynamic viscosity for continuous hybrid/composite hydrogel R217-0.2 and for two-phase hybrid/composite hydrogel #1 (R217-0.2/R40-0.2) at day 4 after storage at room temperature (25° C.).

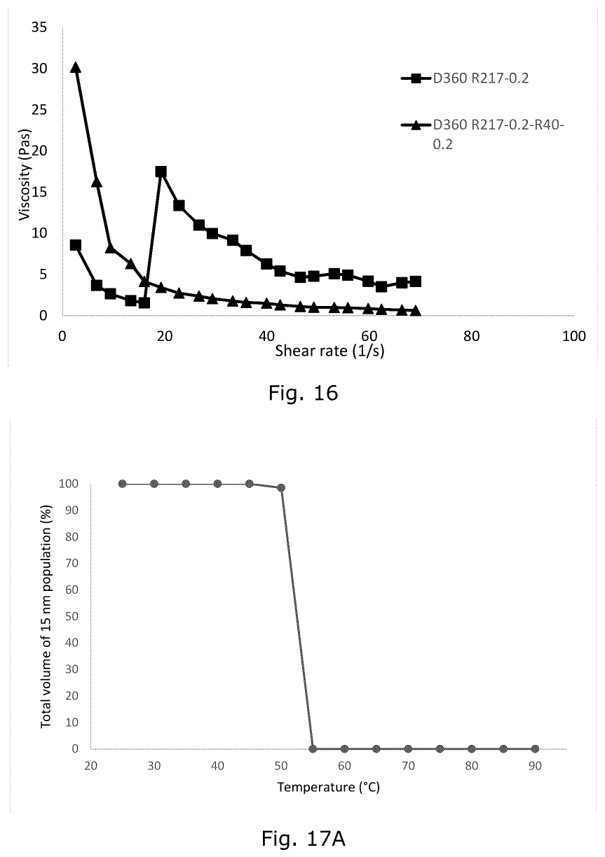

FIG. 16 shows the dynamic viscosity for continuous hybrid/composite hydrogel R217-0.2 and for two-phase hybrid/composite #1 (R217-0.2/R40-0.2) at day 360 after storage in sealed aluminium foil bags at room temperature.

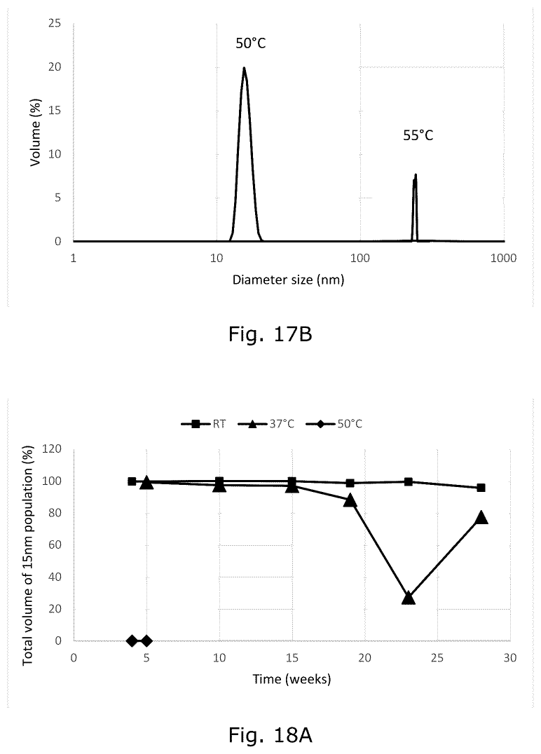

Rheological measurements show that the studied hybrid/composite hydrogels retain their rheological properties quite well after 28 days of storage in syringes enclosed in an aluminium foil bags at 25° C. The damping factor (also called loss tangent or loss factor), which is the ratio between loss modulus and storage modulus (G″/G′) indicates the viscoelastic properties of the hybrid/composite hydrogels at rest (e.g., in syringe). The damping factor results show that the hydrogels retain their structure during the storage and there is no significant change in the properties after 28 days of storage at 25° C. (FIG. 13). The dynamic viscosity (FIG. 16) simulates the injection from syringe, and both studied hydrogels are clearly shear-thinning after 4 days of storage at 25° C. The properties remain relatively unchanged over the period of 1 year (FIGS. 14 and 17). The differences occur primarily in the dynamic viscosity. Overall, the minimum viscosity at small shear rates has dropped drastically and the rate at which the material becomes shear-thinning is slower compared to the D4 and D28 cases. Overall behaviour had not changed; the material was able to retain its rheological properties during storage.

### Injectability

In addition to rheological measurements, the injectability and stability of the hybrid/composite hydrogels were evaluated by injecting the material through 25 G needles from a 1 ml syringe. Evaluation of the injectability or possible phase separation was estimated according to criteria from 0 to 5 for continuous hybrid/composite hydrogel with embedded P-particles R217-0.2 (AG) and for two versions two-phase hybrid/composite hydrogel #1 with embedded P-particles (R217-0.2-R40-0.2 (AG) and R217-0.2 (AG)-R40-0.2 (AG)) at day 4, 28, 180 and 330 after storage in syringes enclosed in an aluminium foil bags at room temperature (25° C.) (Table 6). For smoothness of injection the criteria are defined as follows; 5=very smooth, 4=mostly smooth, 3=fairly smooth, 2=partly smooth, 1=heterogeneous, not at all smooth, 0=not injectable. For phase separation the criteria are defined as follows; 5=no phase separation, 4=minimal phase separation, 3=phase separation, but reversible after injection, 2=phase separation, and irreversible after injection, 1=clear phase separation, 0=not a gel at all.

Table 6 shows the injection experiments for smoothness of injection and phase separation at different time points after storage at 25 C. The score is the average of three replicate injections.

TABLE 6

| | day 4 | day 28 | day 180 | day 330 |
|---|---|---|---|---|
| Smoothness | | | | |
| R217-0.2 (AG) | 5 | 5 | 5 | 5 |
| R217-0.2- | 4 | 4 | 4 | 4 |
| R40-0.2 (AG) | | | | |
| R217-0.2 (AG)- | 3 | 2 | 3 | 3 |
| R40-0.2 (AG) | | | | |
| Phase separation | | | | |
| R217-0.2 (AG) | 4 | 4 | 4 | 4 |
| R217-0.2- | 3 | 3 | 3 | 3 |
| R40-0.2 (AG) | | | | |
| R217-0.2 (AG)- | 4 | 3 | 4 | 4 |
| R40-0.2 (AG) | | | | |

The injection experiments show that smoothness observed at day 4 does not change during the storage, and the small changes observed between the continuous hybrid/composite hydrogel and two-phase hybrid/composite hydrogel #1 originate from the difference in structure, i.e., from the separate gel particles that are present in both versions of two-phase hybrid/composite hydrogel #1. They are all injectable, but the results suggest that R217-0.2 (AG)-R40-0.2 (AG) with P-particles encapsulated both in the continuous phase and dispersed phase is a bit more heterogenous in nature, but still injectable. The amount of encapsulated antigen is very low, and hence it is not probable that it has as such an influence, but rather the small difference in the preparation process (addition of P-particles in R217-0.2), which emphasises the need for strict control of the preparation process. The minimal or fair phase separation observed is related to small amount water coming out first when starting the injection, or an observation of hydrogel lumps in connection with the injection. The gel lumps, however, form a unified hydrogel compact again after the injection.

### P-Particles Form Aggregates in Solution at Elevated Temperatures

As previously shown (Journal of Virological Methods 179 (2012) 1- 7), noroviral P-particles have a diameter approximate of 15-17 nm in solution when analysed with DLS (not shown). In order to study the thermal stability of P-particles we first determined the temperature at which they become aggregated in solution. According to this analysis a sharp temperature-induced aggregation occurred temperatures above 50° C. and all P-particles were found to form aggregates ranged 300 to 830 nm at 55° C. (FIG. 17). Based on these results, we decided to perform the long-term storage and dissolution experiments using the following temperatures: RT, 37° C. and 50° C.

FIGS. 17 show the thermal stability of P-particles measured with DLS. In FIG. 17A, a proportion of 16 nm particles at different temperatures, in FIG. 17B, the difference in the distribution by volume for P-particles measured at 50° C. and 55° C. Both FIGS. 17A and 17B show the average of three separate measurements.

Particle Integrity after Dissolution

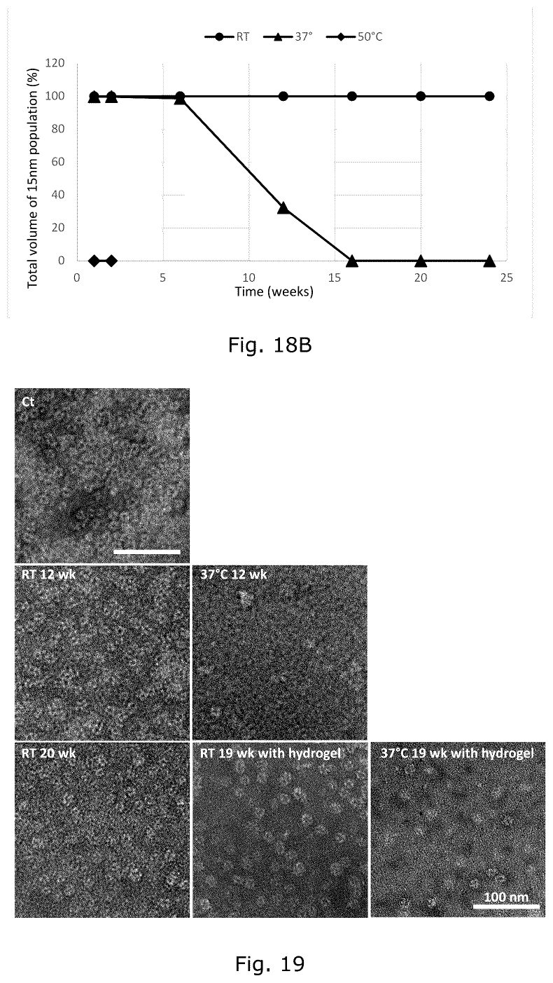

The integrity of the particles dissolved from hydrogel were studied with DLS. During 28-week follow-up practically all particles released from hydrogel kept at RT remained at the expected size and also particles released from hydrogels kept at 37° C. had an expected range of 15-17 nm radius up to 19 weeks (FIG. 18). Control samples in solution kept at RT remained intact but those kept at 37° C. started to destroy after six week and they were totally destroyed within 16 weeks. Again, both the hydrogel and solution samples kept at 50° C. were destroyed within 4 or 2 weeks, respectively.

FIGS. 18 show the total volume of 16 nm population of P-particles either released from continuous hybrid/composite hydrogel R217-0.2 (FIG. 18A) or from control particles (FIG. 18B) kept at solution at indicated temperatures as determined with DLS.

### TEM-Imaging

Transmission electron microscope images show that hydrogel protects P-particles at 37° C. longer than control particles kept at the same temperature in solution.

TEM shows that control P-particles have a typical shape and expected size of approximate 17-20 nm (FIG. 19; Upper panel). P-particles stay stable at RT for 20 weeks in solution (FIG. 19, left lower panel), whereas they seems to disappear when kept at 37° C. for 12 weeks in solution (FIG. 19; middle panel). P-particles dissolved from hydrogel after kept at RT and 37° C. seems to be intact after 19 weeks (FIG. 19; middle lower panel).

### Endotoxin Levels

Endotoxins levels were determined to confirm removal of endotoxins from the antigen. No residual impurities were detected in the purified P-particles, as antigens were free of bacterial endotoxins (<0.012 EU/10 μg protein).

### Development of Serum IgG Antibodies

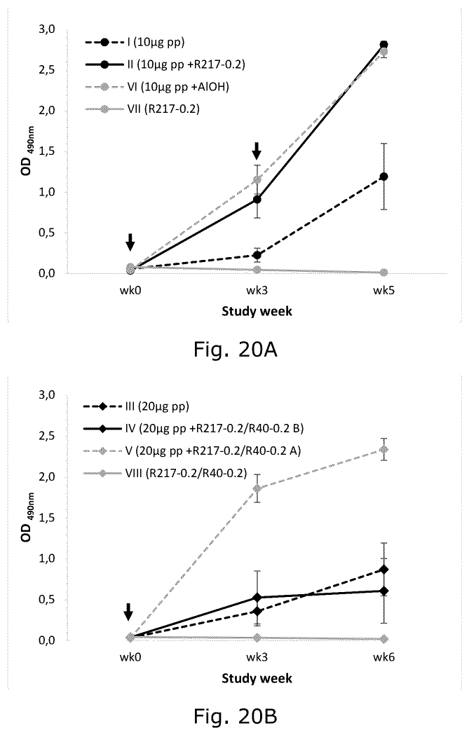

To examine possibility of the present hydrogels to function as an adjuvant/delivery system, mice were immunised with two 10 μg doses of P-particles alone or embedded in continuous hybrid/composite R217-0.2 hydrogel or a single 20 μg dose of P-particles alone or embedded in in two-phase hybrid/composite #1 R217-0.2/R40-0.2 hydrogel. For comparison, one experimental group received P-particles formulated with Al(OH)₃. FIG. 20 illustrates development of serum IgG antibodies against P-particles at study weeks 0, 3 and 5 or 6. According to the results, immunisation with one dose of P-particles induced a very potent immune response only, when the antigen was co-administered with R217-0.2 hydrogel or R217-0.2/R40-0.2 A hydrogel (25 wt-% antigen in R217-0.2). This demonstrates that an increase in antibody responses at week 3 after the single immunisation originates from R217-0.2 component, not from R40-0.2. The second dose of P-particles with R217-0.2 hydrogel elevated remarkably the already induced responses.

FIGS. 20 shows the kinetics of serum IgG antibodies in mice following immunisation with two 10 μg doses of P-particles (pp) alone or formulated with R217-0.2 hydrogel (FIG. 20A) or a single 20 μg dose of P-particles alone or formulated with R217-0.2/R40-0.2 hydrogel (FIG. 20B). Control mice received P-particles with Al(OH)₃ or hydrogels without an antigen. Group mean OD values of tail blood samples and termination sera at indicated study weeks are shown. Immunisation points are indicated with arrows.

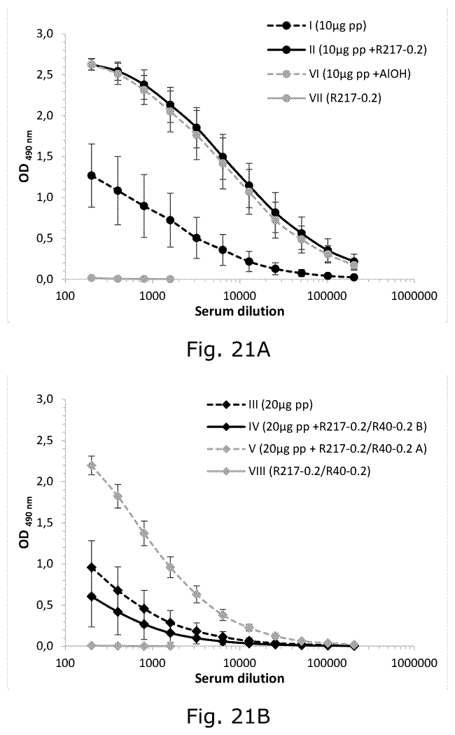

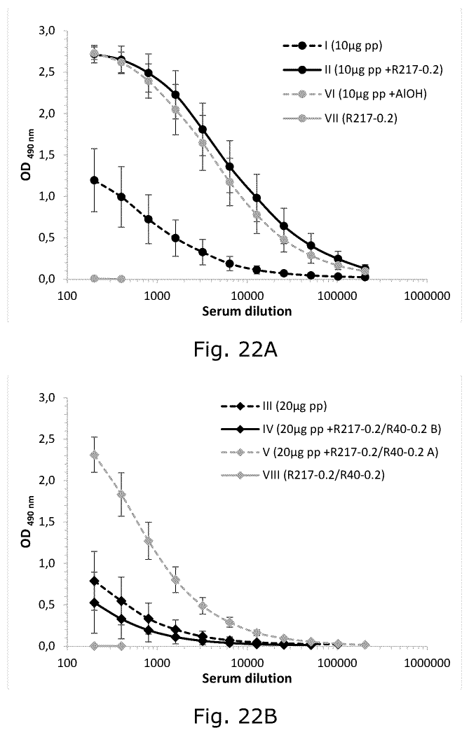

In addition, geometric mean titers were appreciably high (reciprocal titer >4.2 log10) for groups of the mice receiving P-particles with R217-0.2 or R217-0.2/R40-0.2 A hydrogel (25 wt-% antigen in R217-0.2), co-administration resulting in 18- or 10-fold higher levels than those observed with P-particles alone (FIG. 21). This indicates that R217-0.2 increases the magnitude of the antigen-specific IgG response. The observed effect of R217-0.2 on P-particles is however similar to that of Al(OH)₃ hydrogel (FIGS. 21 and 22). Negative control mice had no responses against P-particles.

FIGS. 21A and 21B illustrate endpoint titration of serum IgG in mice following immunisation with two 10 μg doses of P-particles (pp) alone or formulated with R217-0.2 hydrogel (FIG. 21A) or a single 20 μg dose of P-particles alone or formulated with R217-0.2/R40-0.2 hydrogel

(FIG. 21B). Control mice received P-particles with Al(OH)₃ or hydrogels without an antigen. Shown are the mean ODs of the groups.

### Serum Antigen-Specific IgG1 and IgG2a Subtypes

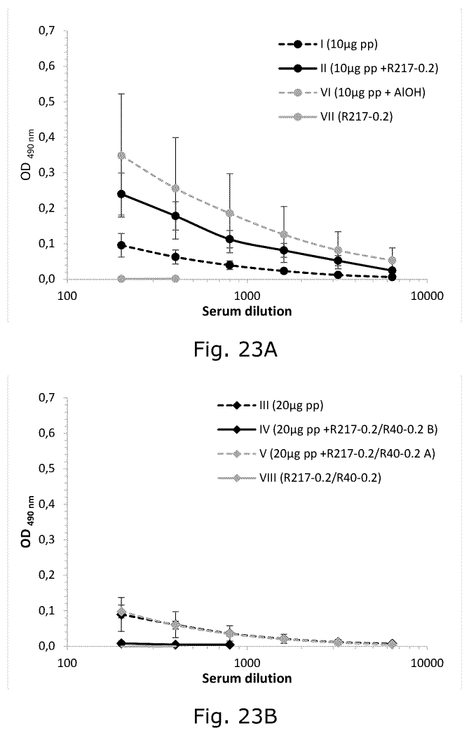

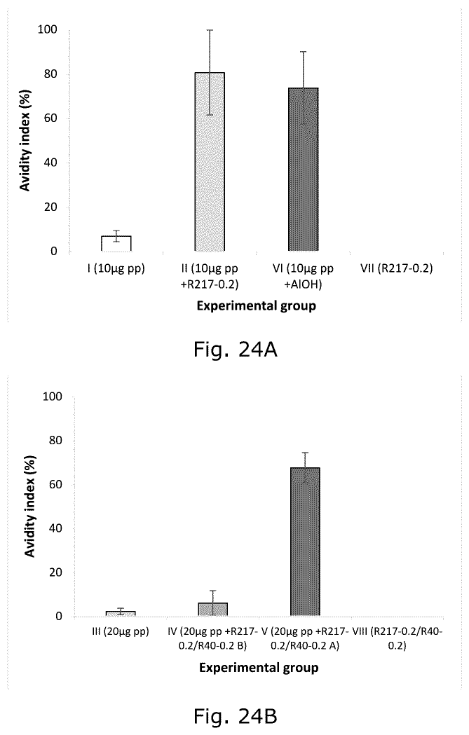

Antigen-specific IgG subtype, IgG1 (a hallmark of a Th2 response) and IgG2a (a hallmark of a Th1 response), analyses showed that only P-particles formulated with continuous hybrid/composite hydrogel R217-0.2 or Al(OH)₃ induce a mixed immune response, namely Th2- and Th1-type (FIGS. 22 and 23). Despite each P-particle formulation induced

Th2 immune responses, co-administration of P-particles with R217-0.2 or two-phase hybrid/composite #1 R217-0.2/R40-0.2 A hydrogels (25 wt-% antigen in R217-0.2) or Al(OH)₃ generated greater levels of IgG1 compared to administration of P-particles alone (FIGS. 22). Instead, a moderate Th1 immune response was induced only by the P-particles co-administered with R217-0.2 hydrogel or Al(OH)₃ (FIGS. 23). These results suggest R217-0.2 to improve the quality of immune responses, functioning preferably as a Th2 adjuvant similar to Al(OH)3.

FIGS. 22 illustrate endpoint titration of serum IgG1 in mice following immunisation with two 10 μg doses of P-particles (pp) alone or formulated with R217-0.2 hydrogel (FIG. 22A) or a single 20 μg dose of P-particles alone or formulated with R217-0.2/R40-0.2 hydrogel (FIG. 22B). Control mice received P-particles with Al(OH)₃ or hydrogels without an antigen. Shown are the mean ODs of the groups.

FIGS. 23 illustrate endpoint titration of serum IgG2a in mice following immunisation with two 10 μg doses of P-particles (pp) alone or formulated with R217-0.2 hydrogel (FIG. 23A) or a single 20 μg dose of P-particles alone or formulated with R217-0.2/R40-0.2 hydrogel (FIG. 23B). Control mice received P-particles with Al(OH)₃ or hydrogels without an antigen. Shown are the mean ODs of the groups.

### Antibody Avidity

Assessment of individual immune sera for the avidity of anti-GII.4 IgG antibodies (FIGS. 24) indicated, that immunisation with P-particles alone induced antibodies with a considerably lower avidity (avidity indices <10%) as compared with antibodies induced with a combination of P-particles and continuous hybrid/composite hydrogel R217-0.2 or two-phase hybrid/composite hydrogel #1 R217-0.2/R40-0.2 A hydrogel (25 wt-% antigen in R217-0.2) or (avidity indices >60%). The same approximate level was elicited by P-particles formulated with Al(OH)3.

According to this analysis R217-0.2 improves the antibody affinity.

FIGS. 24 show avidity of serum IgG antibodies in mice immunised with two 10 μg doses of P-particles (pp) alone or formulated with R217-0.2 hydrogel (FIG. 24A) or a single 20 μg dose of P-particles alone or formulated with R217-0.2/R40-0.2 hydrogel (FIG. 24B). Control mice received P-particles with Al(OH)₃ or hydrogels without an antigen. Mean avidity indices (%) of groups are shown.

### Cross-Reactive Serum IgG Responses

The cross-reactivity of the serum antibodies was measured against four heterologous NoV VLPs derived from genogroup II (GII.4 NO, GII.4

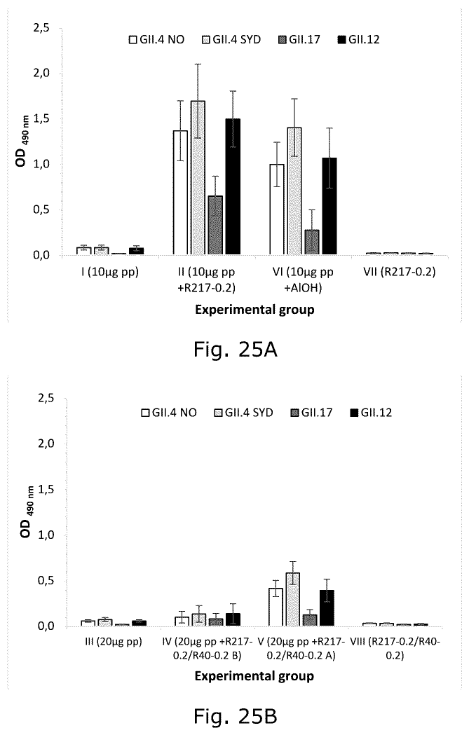

Sydney, GII.12, and GII.17). No cross-reactive IgG antibodies were detected after immunisation with P-particles alone, while formulation with R217-0.2 hydrogel or Al(OH)₃ resulted in antibodies with considerably broader cross-reactivity (FIGS. 25). The results suggest that the increase in cross-reactive antibody responses is derived from R217-0.2 but not from R40-0.2.

FIGS. 25 show cross-reactive serum IgG responses against heterologous NoV VLPs in mice immunised with two 10 μg doses of P-particles (pp) alone or formulated with continuous hybrid/composite R217-0.2 hydrogel (FIG. 25A) or a single 20 μg dose of P-particles alone or formulated with two-phase hybrid/composite #1 R217-0.2/R40-0.2 hydrogel (FIG. 25B). Control mice received P-particles with Al(OH)₃ or hydrogels without an antigen. Shown are the mean ODs of the groups.

### Neutralising Antibodies

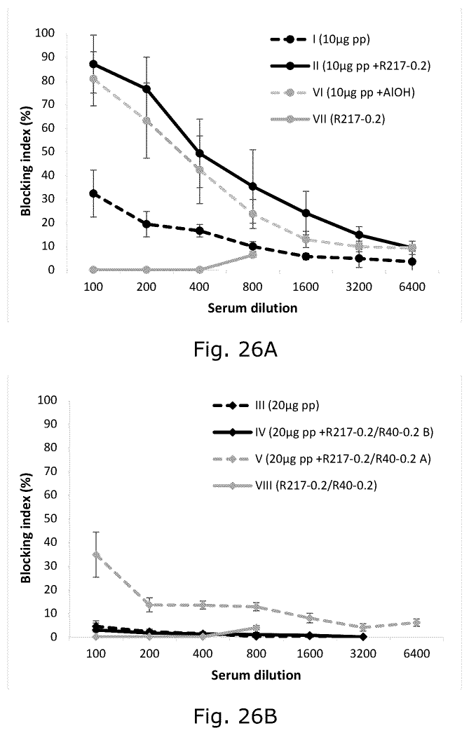

Neutralising ability of induced antibodies was examined measuring blocking activity against homologous GII.4 VLPs with PGM-based blocking assay. Experimental groups receiving 10 μg doses of P-particle formulations or 20 μg dose formulated with two-phase hybrid/composite #1 R217-0.2/R40-0.2 A (25 wt-% antigen in R217-0.2) developed antibodies with detectable blocking ability, co-administration of the antigen with continuous hybrid/composite R217-0.2 hydrogel or Al(OH)₃ elevating the activity significantly (FIGS. 26). Only P-particles formulated with R217-0.2 hydrogel or Al(OH)₃ induced antibodies able to block >50% of the VLP binding. Based on the analysis, strong increase in blocking antibodies originates from formulations with R217-0.2 and Al(OH)₃.

FIGS. 26 show homologous blockage of GII.4 VLP binding to HBGA receptors by serum antibodies of mice immunised with two 10 μg doses of P-particles (pp) alone or formulated with R217-0.2 hydrogel (FIG. 26A) or a single 20 μg dose of P-particles alone or formulated with R217-0.2/R40-0.2 hydrogel (FIG. 26B). Control mice received P-particles with Al(OH)₃ or hydrogels without an antigen. Shown are the mean blocking indices (%) of the groups.

### Mucosal Antibodies

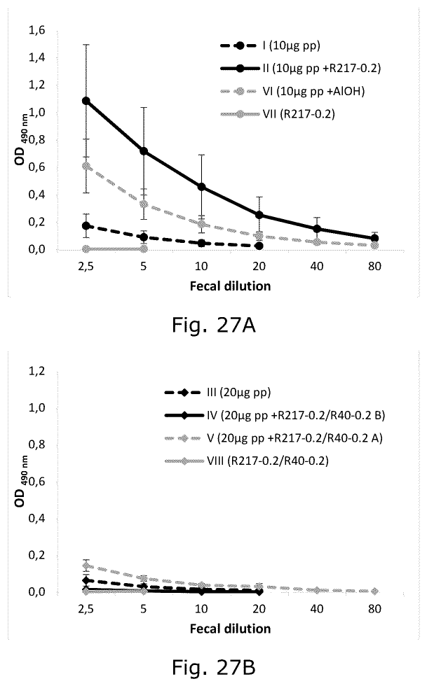

Mucosal antibody analysis demonstrated that immunisation with P-particles alone resulted in extremely low (or negative) levels of faecal antibodies, but formulation of the antigen with continuous hybrid/composite R217-0.2 hydrogel or Al(OH)₃ increased the magnitude of mucosal antibodies (FIGS. 27).

FIGS. 27 illustrate endpoint titrations of faecal IgG antibodies in mice immunised with two 10 μg doses of P-particles (pp) alone or formulated with R217-0.2 hydrogel (FIG. 27A) or a single 20 μg dose of P-particles alone or formulated with two-phase hybrid/composite R217-0.2/R40-0.2 hydrogel (FIG. 27B). Control mice received P-particles with Al(OH)₃ or hydrogels without an antigen. Shown are the mean blocking indices (%) of the groups. Shown are the mean ODs of the groups.

### CONCLUSIONS

The continuous hybrid/composite R217-0.2 hydrogel appears to work as an adjuvant for increasing the magnitude of the serum and mucosal immune responses, and for improving the quality and functionality of the immune responses in terms of antibody avidity, cross-reactivity as well as neutralising antibodies.

The observed adjuvant effect of R217-0.2 is comparable to that of Al(OH)₃ hydrogel. The protein released from the hydrogel resembles original P-particles according to DLS. Furthermore, according to DLS, P-particles aggregate at temperatures 55° C. and above.

It was also noted that 16 nm particles can be released from the hydrogel after storage at RT and 37° C., but not after storage at 50° C., and that the concentration of released particles appears to decrease over time. Compared to storage in solution, the present hydrogel appears to increase the proportion of 15 nm particles at 37° C. after storage time more than five weeks, suggesting stabilisation of the P-particle in the hydrogel.

1. A hydrogel material, comprising a first hydrogel comprising:
0.15-21 wt-% of a functionalised triblock molecule having a formula (1): 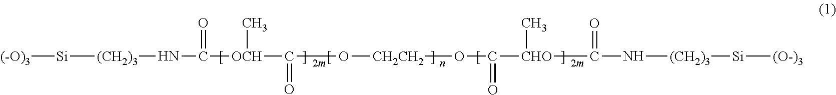
wherein n is 4-680 and m is 1-10, based on a total weight of the first hydrogel,
0.85-4.0 wt-% of silica, based on the total weight of the first hydrogel, and
75-99 wt-% of an aqueous liquid, based on the total weight of the first hydrogel,
wherein the —Si—OH groups of the silica form a —Si—O—Si— bond with the —Si—(O)₃— group of the functionalised triblock molecule of formula (1).

2. The hydrogel material of claim 1, wherein the functionalised triblock molecule of formula (1) and silica form colloidal particles, and a network of said colloidal particles forms a continuous solid phase, in which the aqueous liquid is homogeneously distributed, and the solid phase and aqueous liquid are in a single hydrogel entity.

3. The hydrogel material of claim 1 or 2, further comprising a second hydrogel comprising:
0.15-21 wt-% of the functionalised triblock molecule of formula (1), based on the total weight of the second hydrogel,
0.85-4.0 wt-% silica, based on the total weight of the second hydrogel; and
75-99 wt-% of an aqueous liquid, based on the total weight of the second hydrogel,
wherein the —Si—OH groups of the silica form a —Si—O—Si— bond with the —Si—(O)₃— group of the functionalised triblock molecule of formula (1) of the second hydrogel, and
wherein the second hydrogel is in the form of homogeneously distributed particles dispersed within the first hydrogel, the first hydrogel forming a continuous phase, and
wherein the total amount of silica and functionalised triblock molecule having a formula (1) is greater in the second hydrogel than the total amount of silica and functionalised triblock molecule having a formula (1) in the first hydrogel.

4. The hydrogel material of claim 1, wherein:
the functionalised triblock molecule of formula (1) and a first part of the aqueous liquid are in the form of particles obtained by breaking a hydrogel of the functionalised triblock molecule of formula (1) and the aqueous liquid into particles, dispersed within a silica sol obtained by mixing a second part of the aqueous liquid; and
the —Si—OH groups of the silica have formed —Si—O—Si— bonds with the —Si—(O)₃— group of the functionalised triblock molecule of formula (1), thus forming the hydrogel material.

5. The hydrogel material of claim 1, wherein the silica is an alkoxysilane-derive silica, preferably tetraothoxyi;iiane derived _silica.

6. The hydrogel material of claim 1, wherein the aqueous liquid of the first hydrogel and of the second hydrogel is independently selected from water; a mixture of water and ethanol; and biologically compatible buffers.

7. The hydrogel material of claim 1, wherein the aqueous liquid is a mixture of water and ethanol, comprising 50-95 wt-% of water, the rest being ethanol.

8. The hydrogel material of claim 1, further comprising at least one biologically active agent encapsulated within the first hydrogel.

9. The hydrogel material of claim 3, further comprising at least one biologically active agent encapsulated within at least one of the first hydrogel and the second hydrogel.

10. The hydrogel material of 1, further comprising at least one biologically active agent encapsulated within the hydrogel of the functionalised triblock molecule of formula (1).

11. The hydrogel material of claim 1, further comprising at least one biologically active agent selected from the group consisting of immunomodulatory agents and therapeutically active agents.

12-15. (canceled)

16. The hydrogel material of claim 1, wherein the total amount of silica and functionalised triblock molecule having a formula (1) is at least 20% greater in the second hydrogel than the total amount of silica and functionalised triblock molecule having a formula (1) in the first hydrogel.
